(19) United States
(12) Patent Application Publication
(10) Pub. No.: US 2015/0359794 A1
(43) Pub. Date: Dec. 17, 2015

(54) IMPAIRMENT OF THE LARGE RIBOSOMAL SUBUNIT PROTEIN RPL24 BY DEPLETION OR ACETYLATION

(71) Applicant: Buck Institute for Research on Aging, Novato, CA (US)

(72) Inventors: Christopher C. Benz, Novato, CA (US); Kathleen Wilson-Edell, San Francisco, CA (US); Gary K. Scott, Berkeley, CA (US)

(21) Appl. No.: 14/739,201

(22) Filed: Jun. 15, 2015

Related U.S. Application Data
(60) Provisional application No. 62/030,981, filed on Jul. 30, 2014.
(60) Provisional application No. 62/012,268, filed on Jun. 13, 2014.

(51) Int. Cl. A61K 31/505; A61K 31/506

(52) U.S. Cl. CPC A61K 31/505; A61K 31/506

(57) ABSTRACT

Provided herein are compositions of histone deacetylase (HDAC) inhibitors for the treatment of cancers overexpressing the large ribosomal subunit protein 24 (RPL24) in a subject in need thereof. Provided herein are methods for treating RPL24-overexpressing cancers in a subject in need thereof, comprising administering to the subject an effective amount of an HDAC inhibitor. Also provided herein are methods for inhibiting the viability of an RPL24-overexpressing cancer cell with an HDAC inhibitor. Also provided herein are methods for assessing the efficacy of an HDAC inhibitor against a cancer.

### RELATED APPLICATION

This application is related to U.S. Provisional Application No. 62/030,981, filed Jul. 30, 2014, and U.S. Provisional Application No. 62/012,268, filed Jun. 13, 2014. The entire contents of these applications are incorporated herein by reference in their entirety.

### SEQUENCE LISTING

This application contains a Sequence Listing, which has been submitted electronically in ANSI format and is hereby incorporated by reference in its entirety. Said ANSI copy is named 570311_ACT-024_sequence_listing_ST25.txt and is 5,381 bytes in size.

### TECHNICAL FIELD

Provided herein are treatments for an RPL24-overexpressing cancer by administration of a histone deacetylase (HDAC) inhibitor.

### BACKGROUND

Control of protein synthesis is commonly dysregulated in cancer, most frequently by mutational activation of the phosphoinositide 3-kinase, protein kinase B/Akt/mammalian target of rapamycin (PI3K/Akt/mTOR) pathway. The PI3K/Akt/mTOR pathway promotes cell survival and growth by inducing the phosphorylation of the small (40S) ribosomal subunit protein S6 (RPS6) and the eukaryotic initiation factor 4e binding protein 1 (4eBP1). These events stimulate polysome assembly and increased cap-dependent (eIF4E-dependent) translation of tumorigenic mRNAs. In addition to PI3K/Akt/mTOR, other pathways can cause translational dysregulation in cancer. The large ribosomal subunit protein 24 (RPL24) is one of the later translation factor proteins to be incorporated into the large ribosomal subunit, where it regulates the joining of the 60S subunit to the small 40S subunit. As a translation factor, RPL24 has previously been linked to tumorigenesis, and its functional activity may be modulated by acetylation.

Histone deacetylases are zinc-binding hydrolases that catalyze the deacetylation of lysine residues on histones as well as non-histone proteins. Four families classify the eleven Zn-binding human histone deacetylases identified thus far: Class I (HDAC1, 2, 3 and 8), Class IIa (HDAC4, 5, 7 and 9), Class IIb (HDAC6 and 10), Class III (sirtuins in mammals) and Class IV (HDAC11). HDAC6 is unique among the Zn-dependent histone deacetylases in humans. Located in the cytoplasm, HDAC6 has two catalytic domains and a ubiquitin binding domain in its C-terminal region. Inhibitors of histone deacetylases modulate transcription and induce cell growth arrest, differentiation, and apoptosis. Histone deacetylase inhibitors also enhance the cytotoxic effects of therapeutic agents used in cancer treatment.

Given the prevalence of cancer, and the growing recognition of elevated RPL24 expression associated with them, there is a need for new therapeutic approaches specifically suited for cancers bearing the hallmark of RPL24-overexpression.

### SUMMARY

Provided herein are histone deacetylase (HDAC) inhibitors for the treatment of cancers overexpressing the large ribosomal subunit protein 24 (RPL24) in a subject in need thereof. Also provided herein are methods for inhibiting the viability of an RPL24-overexpressing cancer cell with an HDAC inhibitor. Also provided herein are methods for assessing the efficacy of an HDAC inhibitor against a cancer.

In one aspect, provided herein is a method for treating an RPL24-overexpressing cancer comprising administering an HDAC inhibitor to a subject in need thereof.

In another aspect, provided herein is a method for treating a subject diagnosed with an RPL24-overexpressing cancer comprising administering an HDAC inhibitor to the subject in need thereof.

In one embodiment of these methods, the HDAC is selected from HDAC1, HDAC2, HDAC3, or HDAC8. In another embodiment, the HDAC is selected from HDAC4, HDAC5, HDAC6, HDAC7, HDAC9, or HDAC10. In another embodiment, the HDAC is HDAC11. In another embodiment, the HDAC is HDAC6. In another embodiment, the cancer is a lung cancer. In another embodiment, the cancer is a breast cancer. In another embodiment, the breast cancer is a basal-like breast cancer. In another embodiment, the cancer is an Myc-induced cancer. In another embodiment, the cancer is an Akt-induced cancer.

In another aspect, provided herein is a method for inhibiting the viability of an RPL24-overexpressing cancer cell comprising contacting the cell with an HDAC inhibitor. In one embodiment, the HDAC is selected from HDAC1, HDAC2, HDAC3, or HDAC8. In another embodiment, the HDAC is selected from HDAC4, HDAC5, HDAC6, HDAC7, HDAC9, or HDAC10. In another embodiment, the HDAC is HDAC11. In another embodiment, the HDAC is HDAC6. In another embodiment, the cancer cell is a lung cancer cell. In another embodiment, the cancer cell is a breast cancer cell. In another embodiment, the breast cancer cell is a basal-like breast cancer cell. In another embodiment, the cancer cell is an Myc-induced cancer cell. In another embodiment, the cancer cell is an Akt-induced cancer cell.

In yet another aspect, provided herein is a method for assessing the efficacy of an HDAC inhibitor against an RPL24-overexpressing cancer, comprising the steps of: a) administering an HDAC inhibitor to an RPL24-overexpressing cancer cell; b) measuring the amount of RPL24-acetylation after administration of the HDAC inhibitor to the cell; and c) determining that the HDAC inhibitor is efficacious against the RPL24-overexpressing cancer if there is an increase in RPL24 acetylation after administration of the HDAC inhibitor. In one embodiment, RPL24-acetylation is detected by mass spectrometry. In another embodiment, acetylation of residue K27 of RPL24 is measured. In yet another embodiment, acetylation of residue K93 of RPL24 is measured. In still another embodiment, the HDAC is selected from HDAC1, HDAC2, HDAC3, or HDAC8. In another embodiment, the HDAC is selected from HDAC4, HDAC5, HDAC6, HDAC7, HDAC9, or HDAC10. In another embodiment, the HDAC is HDAC11. In another embodiment, the HDAC is HDAC6. In another embodiment, the cancer is a lung cancer. In another embodiment, the cancer is a breast cancer. In another embodiment, the breast cancer is a basal-like breast cancer. In another embodiment, the cancer is an Myc-induced cancer. In another embodiment, the cancer is an Akt-induced cancer.

In one embodiment, the HDAC inhibitor is a compound of formula IV:

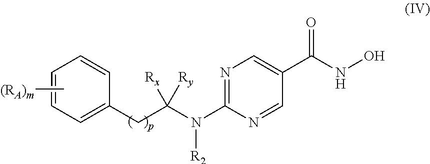

or a pharmaceutically acceptable salt thereof.

In another embodiment, the HDAC inhibitor is the compound:

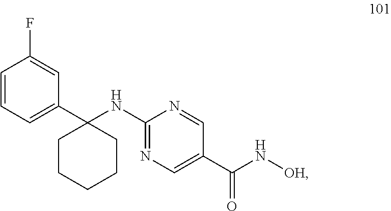 [SMILES: O=C(NO)c1cnc(NC2(c3cccc(F)c3)CCCCC2)nc1]

or a pharmaceutically acceptable salt thereof.

In another aspect, provided herein is a method for treating a subject diagnosed with an RPL24-overexpressing cancer comprising administering the HDAC inhibitor

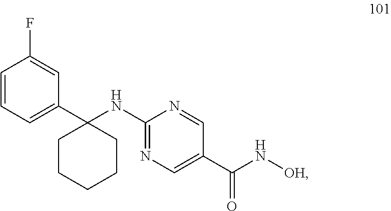 [SMILES: O=C(NO)c1cnc(NC2(c3cccc(F)c3)CCCCC2)nc1]

or a pharmaceutically acceptable salt thereof, to the subject in need thereof.

In yet another aspect, provided herein is a method for inhibiting the viability of an RPL24-overexpressing cancer cell comprising contacting the cell with the HDAC inhibitor

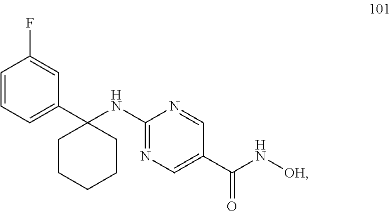 [SMILES: O=C(NO)c1cnc(NC2(c3cccc(F)c3)CCCCC2)nc1]

or a pharmaceutically acceptable salt thereof.

In still another aspect, provided herein is a method for assessing the efficacy of an HDAC inhibitor against an RPL24-overexpressing cancer, comprising the steps of:
a) administering an HDAC inhibitor to the RPL24-overexpressing cancer cell;
b) measuring the amount of RPL24-acetylation after administration of the HDAC inhibitor to the cell; and
c) determining that the HDAC inhibitor is efficacious against the RPL24-overexpressing cancer if there is an increase in RPL24 acetylation after administration of the HDAC inhibitor;
   wherein the HDAC inhibitor is the compound

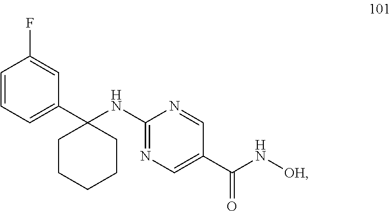 [SMILES: O=C(NO)c1cnc(NC2(c3cccc(F)c3)CCCCC2)nc1]

or a pharmaceutically acceptable salt thereof.

In an embodiment of any one of the methods provided herein, the HDAC inhibitor is an HDAC-selective inhibitor. In an embodiment, the HDAC-selective inhibitor is HDAC1-selective, HDAC2-selective, HDAC3-selective, or HDAC8-selective. In another embodiment, the HDAC-selective inhibitor is HDAC4-selective, HDAC5-selective, HDAC6-selective, HDAC7-selective, HDAC9-selective, or HDAC10-selective. In another embodiment, the HDAC-selective inhibitor is HDAC11-selective. In another embodiment, the HDAC-selective inhibitor is HDAC6-selective.

### BRIEF DESCRIPTION OF DRAWINGS

FIG. 1, panel a, shows RPL24 expression levels in patient-matched breast carcinoma and normal breast tissues.

FIG. 1, panel b, shows differences in RPL24 expression levels between each breast carcinoma and normal breast sample pair.

FIG. 2, panel a, shows that RPL24 knockdown inhibits cap (eIF4E)-dependent expression of proliferation, survival and genome stability proteins.

FIG. 2, panel b, shows that RPL24 knockdown reduces breast cancer cell viability.

FIG. 3, panel a, shows a Western Blot assessment of RPL24 knockdown efficiency in SKBR3 cells.

FIG. 3, panel b, shows RPL24 knockdown reduces 80S and polysome assembly.

FIG. 3, panel c, shows a visualization, with Pymol software, of the location of RPL24 (blue) relative to eIF6 (green) on the previously published structure of the 60S subunit in complex with eIF6.

FIG. 3, panel d, shows RPL24 knockdown increases 60S retention of eIF6.

FIG. 4, panel a, shows that ribosomal protein acetylation is induced by histone deacetylase inhibition as observed by western blots performed in ribopellets, total cytoplasmic lysates, or nuclear extracts.

FIG. 4, panel b, shows that ribosomal protein acetylation is induced by histone deacetylase inhibition as observed by mass spectrometry performed on ribopellets.

FIG. 4, panel c, shows that ribosomal protein acetylation is induced by histone deacetylase inhibition.

FIG. 4, panel d, shows that ribosomal protein acetylation is induced by histone deacetylase inhibition with an HDAC6 siRNA.

FIG. 5, panel a, shows that, like RPL24 knockdown, histone deacetylase inhibition reduces 80S assembly.

FIG. 5, panel b, shows that, like RPL24 knockdown, histone deacetylase inhibition increases 60S retention of eIF6.

FIG. 5, panel c, shows that, like RPL24 knockdown, histone deacetylase inhibition reduces expression of cap (eIF4)-dependently translated proteins.

FIG. 6, panel a, shows a schematic of mass-spectrometry-based techniques to analyze ribosomal protein acetylation.

FIG. 6, panel b, shows the fold change in induction of RPL24 acetylation on K27 by TSA (1 μM, 2 hr) on the 60S subunit and polysomes.

FIG. 6, panel c, shows the fold change in induction of RPL24 acetylation on K93 by TSA (1 μM, 2 hr) on the 60S subunit and polysomes.

FIG. 7, panel a, shows a magnified portion of the RPL24 (blue)-eIF6 (green) interface, visualized with Pymol software, from previous x-ray crystallography data.

FIG. 7, panel b, shows a schematic for modulation of ribosome assembly by RPL24 acetylation.

FIG. 8, panel a, shows a schematic of either full length (amino acids 1-154) or truncated RPL24 (amino acids 1-137).

FIG. 8, panel b, shows polysome profiles two days following transfection of 293T cells with either full length (amino acids 1-154) or truncated RPL24 (amino acids 1-137).

FIG. 8, panel c, shows that expression of truncated RPL24 increases association of eIF6 with 60S fractions in 293T cells.

FIG. 9 shows that TSA-induced HER2 mRNA decay is abrogated by cycloheximide treatment.

FIG. 10 shows ESI-MS/MS spectra for lysine acetylated peptide (SEQ ID NO:7) obtained from polysome preparations.

FIG. 11 shows ESI-MS/MS spectra for lysine acetylated peptide (SEQ ID NO:8) obtained from polysome preparations.

FIG. 12 shows ESI-MS/MS spectra for lysine acetylated peptide (SEQ ID NO:9) obtained from polysome preparations.

FIG. 13 shows ESI-MS/MS spectra for lysine acetylated peptide (SEQ ID NO:3) obtained from polysome preparations.

FIG. 14 shows ESI-MS/MS spectra for lysine acetylated peptide (SEQ ID NO:10) obtained from polysome preparations.

FIG. 15 shows ESI-MS/MS spectra for lysine acetylated peptide (SEQ ID NO:11) obtained from polysome preparations.

FIG. 16 shows ESI-MS/MS spectra for lysine acetylated peptide (SEQ ID NO:12) obtained from polysome preparations.

FIG. 17 shows ESI-MS/MS spectra for lysine acetylated peptide (SEQ ID NO:13) obtained from polysome preparations.

FIG. 18 shows ESI-MS/MS spectra for lysine acetylated peptide (SEQ ID NO:14) obtained from polysome preparations.

FIG. 19 shows ESI-MS/MS spectra for lysine acetylated peptide (SEQ ID NO:15) obtained from polysome preparations.

FIG. 20 shows ESI-MS/MS spectra for lysine acetylated peptide (SEQ ID NO:16) obtained from polysome preparations.

FIG. 21 shows ESI-MS/MS spectra for lysine acetylated peptide (SEQ ID NO:17) obtained from polysome preparations.

FIG. 22 shows ESI-MS/MS spectra for lysine acetylated peptide (SEQ ID NO:18) obtained from polysome preparations.

FIG. 23 shows ESI-MS/MS spectra for lysine acetylated peptide (SEQ ID NO:19) obtained from polysome preparations.

FIG. 24 shows ESI-MS/MS spectra for lysine acetylated peptide (SEQ ID NO:20) obtained from polysome preparations.

FIG. 25 shows ESI-MS/MS spectra for lysine acetylated peptide (SEQ ID NO:21) obtained from polysome preparations.

FIG. 26 shows ESI-MS/MS spectra for lysine acetylated peptide (SEQ ID NO:22) obtained from polysome preparations.

FIG. 27 shows ESI-MS/MS spectra for lysine acetylated peptide (SEQ ID NO:1) obtained from 60S preparations.

FIG. 28 shows ESI-MS/MS spectra for lysine acetylated peptide (SEQ ID NO:3) obtained from 60S preparations.

### DETAILED DESCRIPTION

Provided herein are histone deacetylase (HDAC) inhibitors for the treatment of cancers overexpressing the large ribosomal subunit protein 24 (RPL24) in a subject in need thereof. Also provided herein are methods for inhibiting the viability of an RPL24-overexpressing cancer cell with an HDAC inhibitor. Also provided herein are methods for assessing the efficacy of an HDAC inhibitor against a cancer. In some embodiments, the cancer is lung, breast, basal-like breast, Myc-induced or Akt-induced.

As shown herein, human breast cancers express significantly more RPL24 than matched normal samples. Depletion of RPL24 protein by ≧70% in SKBR3 cells reduce viability by 80% and decrease protein expression of cyclin D1 (75%), survivin (46%) and NBS1 (30%) without altering GAPDH or beta-tubulin levels. Furthermore, as shown herein, RPL24 knockdown reduces 80S subunit levels relative to 40S and 60S levels, and increases 60S retention of the anti-assembly factor eIF6, effects mimicked by 2-24 h treatment with a pan-histone deacetylase inhibitor. The pan-histone deacetylase trichostatin-A, as shown herein, induces acetylation of 15 different polysome-associated proteins including RPL24. K27 is identified as the site of RPL24 acetylation associated with impaired 60S to 80S maturation. HDAC6-selective inhibition or its knockdown similarly induces ribosomal acetylation. As shown herein, histone deacetylase inhibitor treatment does not alter RPL24 levels but induces RPL24 K27 acetylation within the 60S subunit, and also mimics the RPL24 depletion effects. The most notable effect is a markedly reduced viability of oncogenic cells. The results herein demonstrate histone deacetylase inhibition with a compound of formula IV can treat RPL24-overexpressing cancer.

Accordingly, in one aspect, provided herein is a method for treating a subject diagnosed with an RPL24-overexpressing cancer comprising administering an HDAC inhibitor to the subject in need thereof. In one embodiment, the HDAC is selected from HDAC1, HDAC2, HDAC3, or HDAC8. In another embodiment, the HDAC is selected from HDAC4, HDAC5, HDAC6, HDAC7, HDAC9, or HDAC10. In another embodiment, the HDAC is HDAC11. In another embodiment, the HDAC is HDAC6. In another embodiment, the cancer is a lung cancer. In another embodiment, the cancer is a breast cancer. In another embodiment, the breast cancer is a basal-like breast cancer. In another embodiment, the cancer is an Myc-induced cancer. In another embodiment, the cancer is an Akt-induced cancer.

In another aspect, provided herein is a method for inhibiting the viability of an RPL24-overexpressing cancer cell comprising contacting the cell with an HDAC inhibitor. In one embodiment, the HDAC is selected from HDAC1, HDAC2, HDAC3, or HDAC8. In another embodiment, the HDAC is selected from HDAC4, HDAC5, HDAC6, HDAC7, HDAC9, or HDAC10. In another embodiment, the HDAC is HDAC11. In another embodiment, the HDAC is HDAC6. In another embodiment, the cancer cell is a lung cancer cell. In another embodiment, the cancer cell is a breast cancer cell. In another embodiment, the breast cancer cell is a basal-like breast cancer cell. In another embodiment, the cancer cell is an Myc-induced cancer cell. In another embodiment, the cancer cell is an Akt-induced cancer cell.

### Compounds

Provided herein are methods of treatment comprising administration of an HDAC inhibitor.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “HDAC-selective” means that the compound binds to an HDAC to a substantially greater extent, such as 5×, 10×, 15×, 20× greater or more, than to any other type of HDAC enzyme. For example, a compound that binds to HDAC1 and HDAC2 with an IC₅₀ of 10 nM and to HDAC3 with an IC₅₀ of 50 nM is HDAC1/2-selective. On the other hand, a compound that binds to HDAC1 and HDAC2 with an IC₅₀ of 50 nM and to HDAC3 with an IC₅₀ of 60 nM is not HDAC1/2-selective.

The term “inhibitor” is synonymous with the term antagonist.

As used herein, histone deacetylase inhibition refers to the inhibition of an activity of a histone deacetylase. In certain embodiments, histone deacetylase inhibition refers to the inhibition of an activity of histone deacetylase by a compound of formula IV as described below.

Provided herein are methods of treatment comprising administration of an HDAC inhibitor of formula IV:

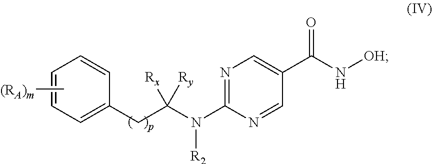

or a pharmaceutically acceptable salt thereof,

wherein,

R₂ is H or alkyl;

Rₓ and R_{y} are independently H, alkyl, or aryl, wherein the alkyl and aryl groups may be substituted with halo; or Rₓ and R_{y} together with the carbon to which each is attached, forms a cycloalkyl or heterocycloalkyl ring;

each R_{A} is independently alkyl, alkoxy, aryl, halo, or haloalkyl; or two R_{A} groups, together with the atoms to which each is attached, can form a heterocycloalkyl ring;

m is 0, 1, or 2; and

p is 0 or 1.

In one embodiment, R₂ is H;

Rₓ and R_{y} are independently H, alkyl, aryl, or haloaryl; or Rₓ and R_{y} together with the carbon to which each is attached, forms a cycloalkyl or heterocycloalkyl ring;

each R_{A} is independently alkyl, alkoxy, aryl, halo, or haloalkyl; or two R_{A} groups, together with the atoms to which each is attached, can form a heterocycloalkyl ring;

m is 0, 1, or 2; and

p is 0.

In another embodiment, Rₓ and R_{y}, together with the carbon to which each is attached, forms a cyclopropyl, cyclobutyl, cyclopentyl, cyclohexyl, oxetanyl, or tetrahydropyranyl ring.

In another embodiment, Rₓ and R_{y}, together with the carbon to which each is attached, forms a cyclopropyl, cyclopentyl, cyclohexyl, or tetrahydropyran ring.

In another embodiment, Rₓ and R_{y}, together with the carbon to which each is attached, forms a cyclopropyl or cyclohexyl ring.

In another embodiment, m is 0, 1 or 2, and each R_{A} is independently methyl, phenyl, F, Cl, methoxy, or CF₃; or two R_{A} groups, together with the atoms to which each is attached, form a dioxole ring.

In another embodiment, m is 1, and R_{A} is F, Cl, methoxy, or CF₃.

Representative compounds of formula IV include, but are not limited to, the following compounds of Table 1 below, or pharmaceutically acceptable salts thereof.

TABLE 1

| | |
|---|---|
| 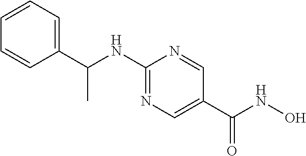 [SMILES: CC(Nc1ncc(C(=O)NO)cn1)c1ccccc1] | 32 |
| 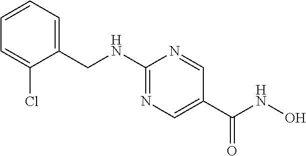 [SMILES: O=C(NO)c1cnc(NCc2ccccc2Cl)nc1] | 33 |
| 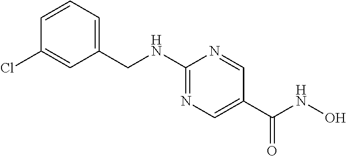 [SMILES: O=C(NO)c1cnc(NCc2cccc(Cl)c2)nc1] | 34 |
| 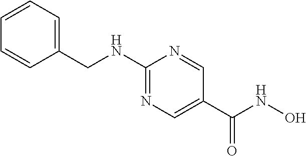 [SMILES: O=C(NO)c1cnc(NCc2ccccc2)nc1] | 35 |
| 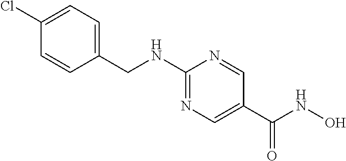 [SMILES: O=C(NO)c1cnc(NCc2ccc(Cl)cc2)nc1] | 36 |
| 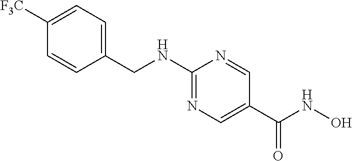 [SMILES: O=C(NO)c1cnc(NCc2ccc(C(F)(F)F)cc2)nc1] | 37 |
| 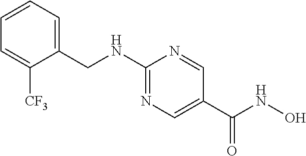 [SMILES: O=C(NO)c1cnc(NCc2ccccc2C(F)(F)F)nc1] | 38 |
| 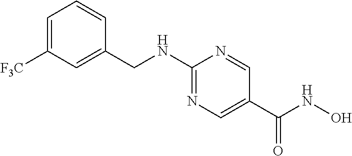 [SMILES: O=C(NO)c1cnc(NCc2cccc(C(F)(F)F)c2)nc1] | 40 |
| 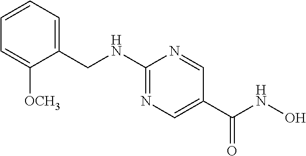 [SMILES: COc1ccccc1CNc1ncc(C(=O)NO)cn1] | 45 |
| 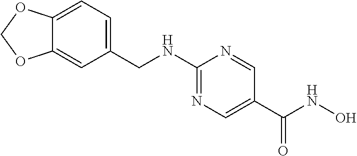 [SMILES: O=C(NO)c1cnc(NCc2ccc3c(c2)OCO3)nc1] | 46 |
| 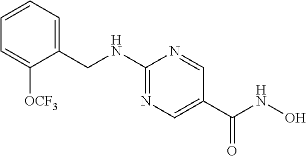 [SMILES: O=C(NO)c1cnc(NCc2ccccc2OC(F)(F)F)nc1] | 47 |
| 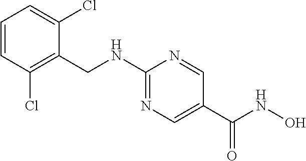 [SMILES: O=C(NO)c1cnc(NCc2c(Cl)cccc2Cl)nc1] | 48 |
| 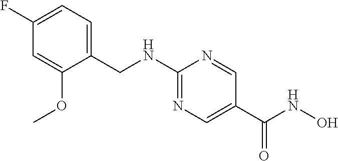 [SMILES: COc1cc(F)ccc1CNc1ncc(C(=O)NO)cn1] | 49 |
| 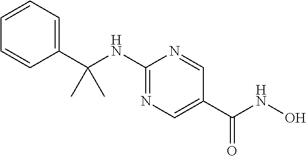 [SMILES: CC(C)(Nc1ncc(C(=O)NO)cn1)c1ccccc1] | 50 |
| 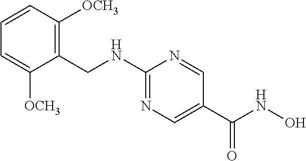 [SMILES: COc1cccc(OC)c1CNc1ncc(C(=O)NO)cn1] | 51 |
| 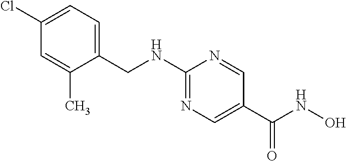 [SMILES: Cc1cc(Cl)ccc1CNc1ncc(C(=O)NO)cn1] | 52 |
| 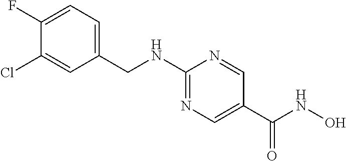 [SMILES: O=C(NO)c1cnc(NCc2ccc(F)c(Cl)c2)nc1] | 53 |
| 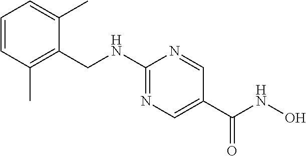 [SMILES: Cc1cccc(C)c1CNc1ncc(C(=O)NO)cn1] | 54 |
| 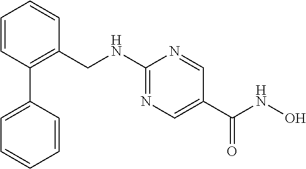 [SMILES: O=C(NO)c1cnc(NCc2ccccc2-c2ccccc2)nc1] | 55 |
| 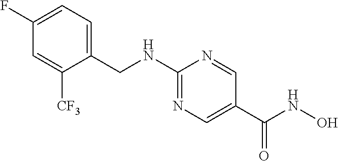 [SMILES: O=C(NO)c1cnc(NCc2ccc(F)cc2C(F)(F)F)nc1] | 57 |
| 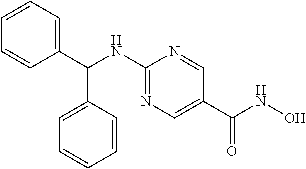 [SMILES: O=C(NO)c1cnc(NC(c2ccccc2)c2ccccc2)nc1] | 60 |
| 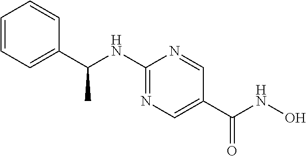 [SMILES: C[C@H](Nc1ncc(C(=O)NO)cn1)c1ccccc1] | 61 |
| 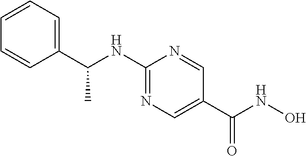 [SMILES: C[C@@H](Nc1ncc(C(=O)NO)cn1)c1ccccc1] | 62 |
| 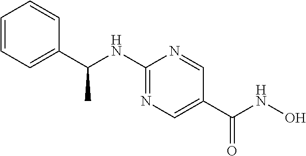 [SMILES: C[C@H](Nc1ncc(C(=O)NO)cn1)c1ccccc1] | 65 |
| 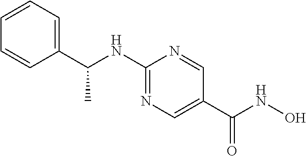 [SMILES: C[C@@H](Nc1ncc(C(=O)NO)cn1)c1ccccc1] | 66 |
| 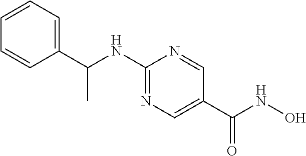 [SMILES: CC(Nc1ncc(C(=O)NO)cn1)c1ccccc1] | 67 |
| 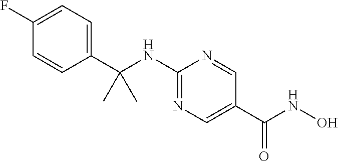 [SMILES: CC(C)(Nc1ncc(C(=O)NO)cn1)c1ccc(F)cc1] | 68 |
| 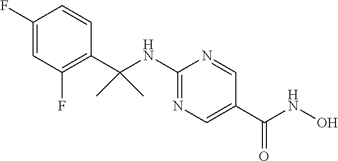 [SMILES: CC(C)(Nc1ncc(C(=O)NO)cn1)c1ccc(F)cc1F] | 70 |
| 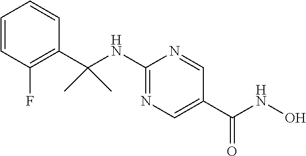 [SMILES: CC(C)(Nc1ncc(C(=O)NO)cn1)c1ccccc1F] | 71 |
| 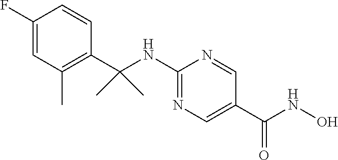 [SMILES: Cc1cc(F)ccc1C(C)(C)Nc1ncc(C(=O)NO)cn1] | 72 |
| 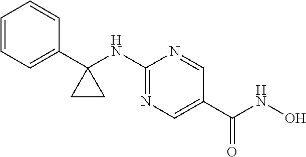 [SMILES: O=C(NO)c1cnc(NC2(c3ccccc3)CC2)nc1] | 73 |
| 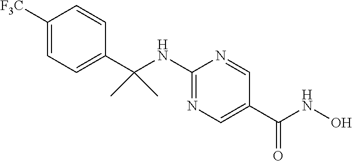 [SMILES: CC(C)(Nc1ncc(C(=O)NO)cn1)c1ccc(C(F)(F)F)cc1] | 74 |
| 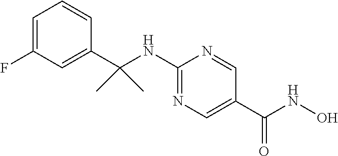 [SMILES: CC(C)(Nc1ncc(C(=O)NO)cn1)c1cccc(F)c1] | 75 |
| 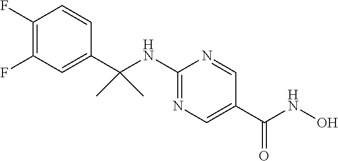 [SMILES: CC(C)(Nc1ncc(C(=O)NO)cn1)c1ccc(F)c(F)c1] | 76 |
| 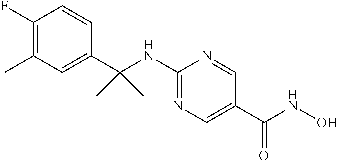 [SMILES: Cc1cc(C(C)(C)Nc2ncc(C(=O)NO)cn2)ccc1F] | 78 |
| 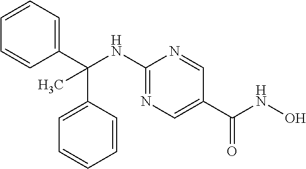 [SMILES: CC(Nc1ncc(C(=O)NO)cn1)(c1ccccc1)c1ccccc1] | 79 |
| 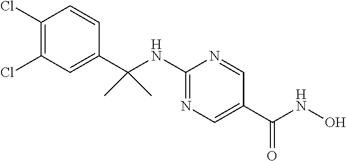 [SMILES: CC(C)(Nc1ncc(C(=O)NO)cn1)c1ccc(Cl)c(Cl)c1] | 80 |
| 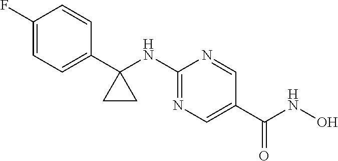 [SMILES: O=C(NO)c1cnc(NC2(c3ccc(F)cc3)CC2)nc1] | 81 |
| 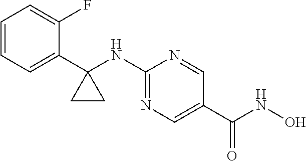 [SMILES: O=C(NO)c1cnc(NC2(c3ccccc3F)CC2)nc1] | 82 |
| 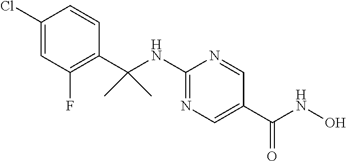 [SMILES: CC(C)(Nc1ncc(C(=O)NO)cn1)c1ccc(Cl)cc1F] | 83 |
| 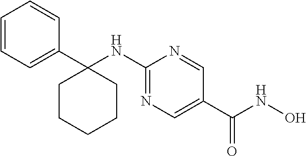 [SMILES: O=C(NO)c1cnc(NC2(c3ccccc3)CCCCC2)nc1] | 84 |
| 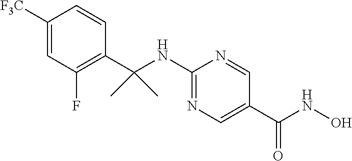 [SMILES: CC(C)(Nc1ncc(C(=O)NO)cn1)c1ccc(C(F)(F)F)cc1F] | 86 |
| 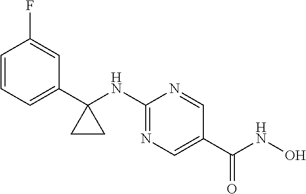 [SMILES: O=C(NO)c1cnc(NC2(c3cccc(F)c3)CC2)nc1] | 87 |
| 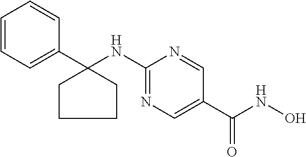 [SMILES: O=C(NO)c1cnc(NC2(c3ccccc3)CCCC2)nc1] | 88 |
| 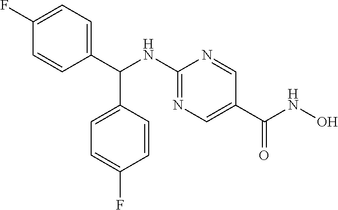 [SMILES: O=C(NO)c1cnc(NC(c2ccc(F)cc2)c2ccc(F)cc2)nc1] | 89 |
| 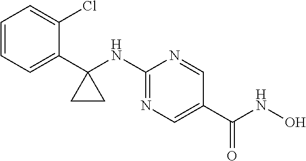 [SMILES: O=C(NO)c1cnc(NC2(c3ccccc3Cl)CC2)nc1] | 90 |
| 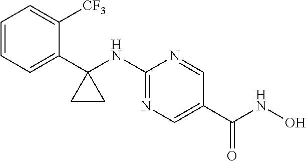 [SMILES: O=C(NO)c1cnc(NC2(c3ccccc3C(F)(F)F)CC2)nc1] | 91 |
| 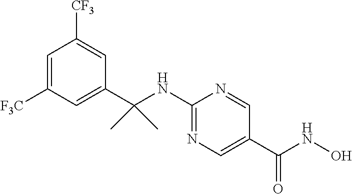 [SMILES: CC(C)(Nc1ncc(C(=O)NO)cn1)c1cc(C(F)(F)F)cc(C(F)(F)F)c1] | 92 |
| 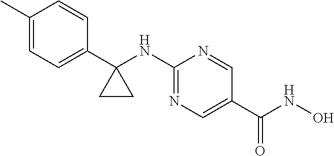 [SMILES: Cc1ccc(C2(Nc3ncc(C(=O)NO)cn3)CC2)cc1] | 93 |
| 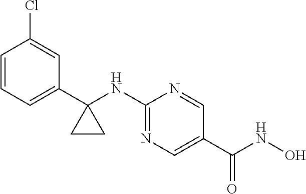 [SMILES: O=C(NO)c1cnc(NC2(c3cccc(Cl)c3)CC2)nc1] | 94 |
| 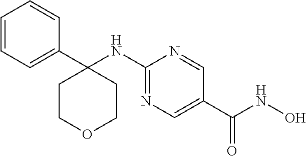 [SMILES: O=C(NO)c1cnc(NC2(c3ccccc3)CCOCC2)nc1] | 95 |
| 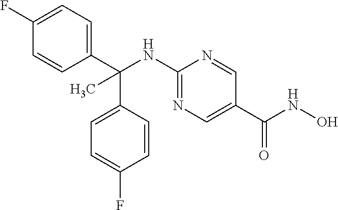 [SMILES: CC(Nc1ncc(C(=O)NO)cn1)(c1ccc(F)cc1)c1ccc(F)cc1] | 96 |
| 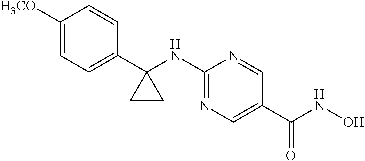 [SMILES: COc1ccc(C2(Nc3ncc(C(=O)NO)cn3)CC2)cc1] | 97 |
| 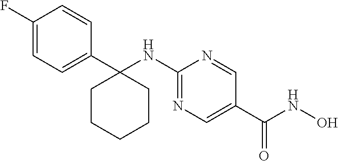 [SMILES: O=C(NO)c1cnc(NC2(c3ccc(F)cc3)CCCCC2)nc1] | 100 |
| 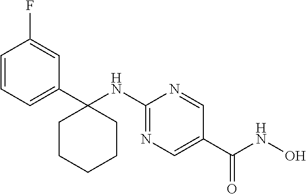 [SMILES: O=C(NO)c1cnc(NC2(c3cccc(F)c3)CCCCC2)nc1] | 101 |
| 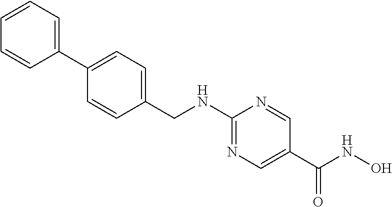 [SMILES: O=C(NO)c1cnc(NCc2ccc(-c3ccccc3)cc2)nc1] | 107 |
| 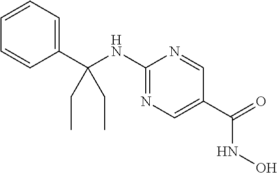 [SMILES: CCC(CC)(Nc1ncc(C(=O)NO)cn1)c1ccccc1] | 113 |
| 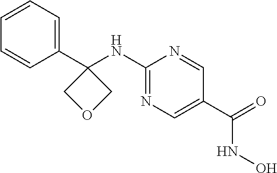 [SMILES: O=C(NO)c1cnc(NC2(c3ccccc3)COC2)nc1] | 114 |
| 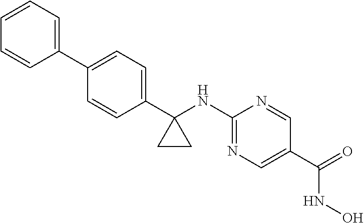 [SMILES: O=C(NO)c1cnc(NC2(c3ccc(-c4ccccc4)cc3)CC2)nc1] | 117 |
| 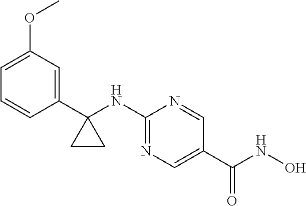 [SMILES: COc1cccc(C2(Nc3ncc(C(=O)NO)cn3)CC2)c1] | 120 |
| 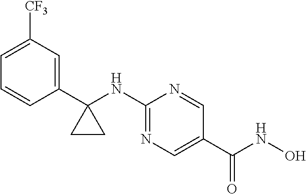 [SMILES: O=C(NO)c1cnc(NC2(c3cccc(C(F)(F)F)c3)CC2)nc1] | 121 |
| 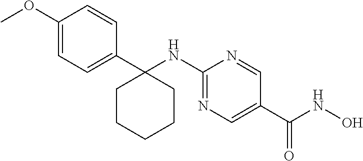 [SMILES: COc1ccc(C2(Nc3ncc(C(=O)NO)cn3)CCCCC2)cc1] | 122 |
| 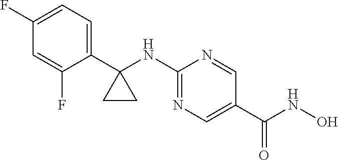 [SMILES: O=C(NO)c1cnc(NC2(c3ccc(F)cc3F)CC2)nc1] | 123 |
| 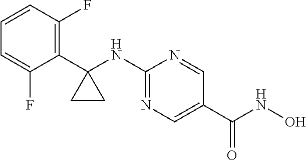 [SMILES: O=C(NO)c1cnc(NC2(c3c(F)cccc3F)CC2)nc1] | 124 |
| 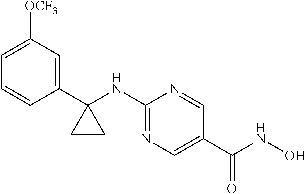 [SMILES: O=C(NO)c1cnc(NC2(c3cccc(OC(F)(F)F)c3)CC2)nc1] | 125 |
| 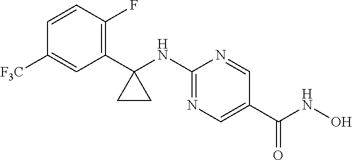 [SMILES: O=C(NO)c1cnc(NC2(c3cc(C(F)(F)F)ccc3F)CC2)nc1] | 126 |
| 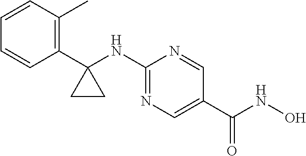 [SMILES: Cc1ccccc1C1(Nc2ncc(C(=O)NO)cn2)CC1] | 127 |
| 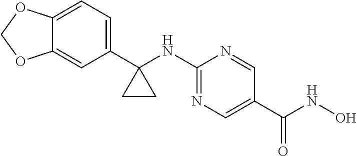 [SMILES: O=C(NO)c1cnc(NC2(c3ccc4c(c3)OCO4)CC2)nc1] | 128 |
| 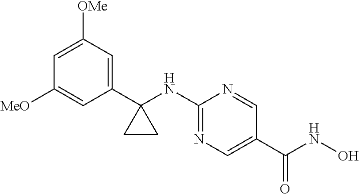 [SMILES: COc1cc(OC)cc(C2(Nc3ncc(C(=O)NO)cn3)CC2)c1] | 129 |
| 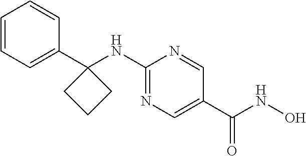 [SMILES: O=C(NO)c1cnc(NC2(c3ccccc3)CCC2)nc1] | 130 |
| 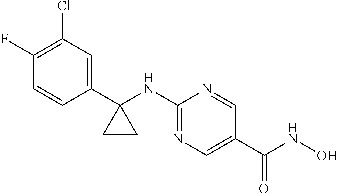 [SMILES: O=C(NO)c1cnc(NC2(c3ccc(F)c(Cl)c3)CC2)nc1] | 131 |
| 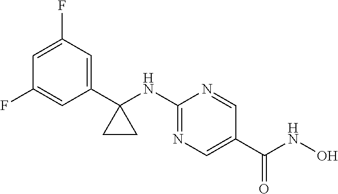 [SMILES: O=C(NO)c1cnc(NC2(c3cc(F)cc(F)c3)CC2)nc1] | 132 |
| 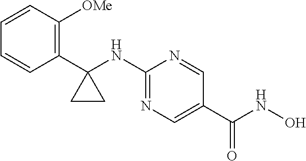 [SMILES: COc1ccccc1C1(Nc2ncc(C(=O)NO)cn2)CC1] | 133 |
| 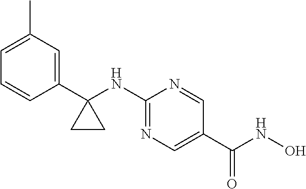 [SMILES: Cc1cccc(C2(Nc3ncc(C(=O)NO)cn3)CC2)c1] | 134 |
| 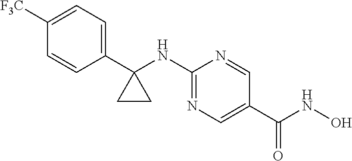 [SMILES: O=C(NO)c1cnc(NC2(c3ccc(C(F)(F)F)cc3)CC2)nc1] | 135 |
| 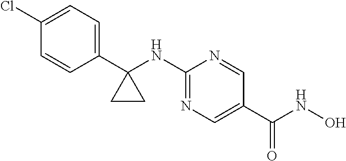 [SMILES: O=C(NO)c1cnc(NC2(c3ccc(Cl)cc3)CC2)nc1] | 136 |
| 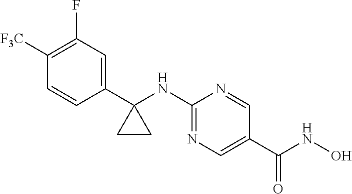 [SMILES: O=C(NO)c1cnc(NC2(c3ccc(C(F)(F)F)c(F)c3)CC2)nc1] | 137 |
| 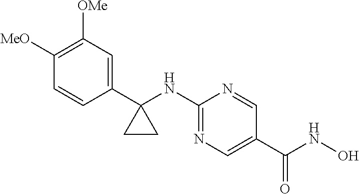 [SMILES: COc1ccc(C2(Nc3ncc(C(=O)NO)cn3)CC2)cc1OC] | 138 |
| 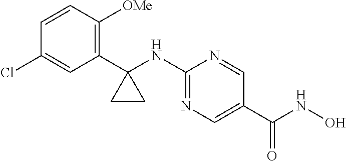 [SMILES: COc1ccc(Cl)cc1C1(Nc2ncc(C(=O)NO)cn2)CC1] | 139 |
| 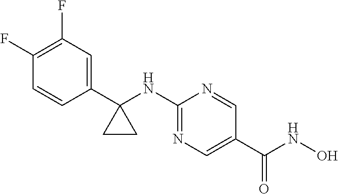 [SMILES: O=C(NO)c1cnc(NC2(c3ccc(F)c(F)c3)CC2)nc1] | 140 |
| 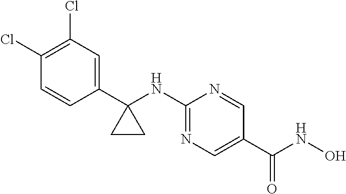 [SMILES: O=C(NO)c1cnc(NC2(c3ccc(Cl)c(Cl)c3)CC2)nc1] | 141 |
| 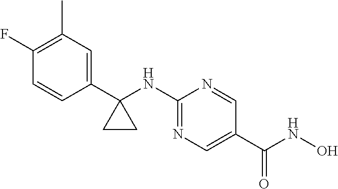 [SMILES: Cc1cc(C2(Nc3ncc(C(=O)NO)cn3)CC2)ccc1F] | 142 |
| 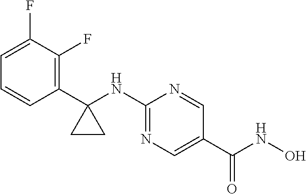 [SMILES: O=C(NO)c1cnc(NC2(c3cccc(F)c3F)CC2)nc1] | 143 |
| 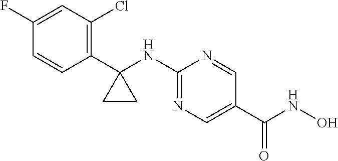 [SMILES: O=C(NO)c1cnc(NC2(c3ccc(F)cc3Cl)CC2)nc1] | 144 |
| 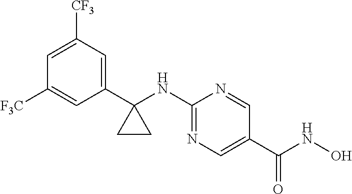 [SMILES: O=C(NO)c1cnc(NC2(c3cc(C(F)(F)F)cc(C(F)(F)F)c3)CC2)nc1] | 145 |
| 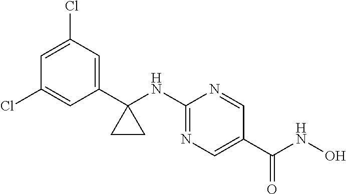 [SMILES: O=C(NO)c1cnc(NC2(c3cc(Cl)cc(Cl)c3)CC2)nc1] | 146 |
| 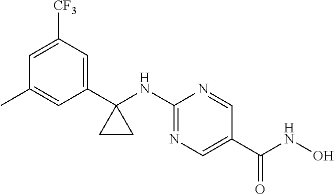 [SMILES: Cc1cc(C(F)(F)F)cc(C2(Nc3ncc(C(=O)NO)cn3)CC2)c1] | 147 |
| 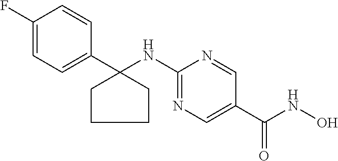 [SMILES: O=C(NO)c1cnc(NC2(c3ccc(F)cc3)CCCC2)nc1] | 148 |
| 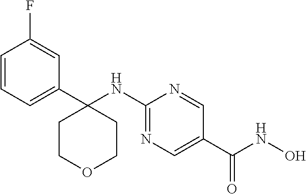 [SMILES: O=C(NO)c1cnc(NC2(c3cccc(F)c3)CCOCC2)nc1] | 149 |
| 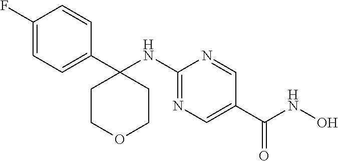 [SMILES: O=C(NO)c1cnc(NC2(c3ccc(F)cc3)CCOCC2)nc1] | 150 |
| 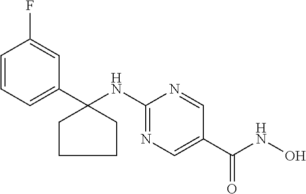 [SMILES: O=C(NO)c1cnc(NC2(c3cccc(F)c3)CCCC2)nc1] | 151 |
| 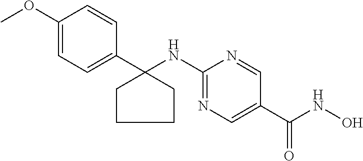 [SMILES: COc1ccc(C2(Nc3ncc(C(=O)NO)cn3)CCCC2)cc1] | 152 |
| 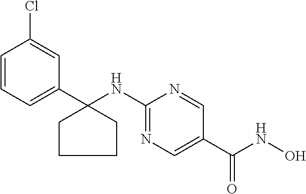 [SMILES: O=C(NO)c1cnc(NC2(c3cccc(Cl)c3)CCCC2)nc1] | 153 |
| 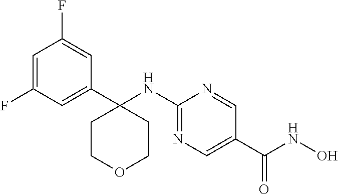 [SMILES: O=C(NO)c1cnc(NC2(c3cc(F)cc(F)c3)CCOCC2)nc1] | 155 |

In a particular embodiment, the compound of formula IV is the compound 101, or a pharmaceutically acceptable salt thereof:

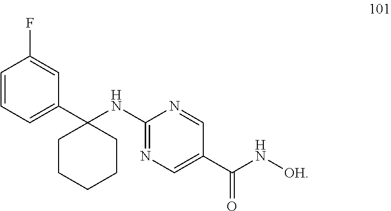 [SMILES: O=C(NO)c1cnc(NC2(c3cccc(F)c3)CCCCC2)nc1]

Also provided herein is a compound as described herein in the manufacture of a medicament for use in the treatment of a disorder or disease herein. Also provided herein is a compound as described herein for use in the treatment of a disorder or disease herein.

Another aspect is an isotopically labeled compound of formula IV delineated herein. Such compounds have one or more isotope atoms which may or may not be radioactive (e.g., ³H, ²H, ¹⁴C, ¹³C, ³⁵S, ³²P, ¹²⁵I, and ¹³¹I) introduced into the compound. Such compounds are useful for drug metabolism studies and diagnostics, as well as therapeutic applications.

Protected derivatives of the compounds provided herein can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999, and subsequent editions thereof.

### Methods

Control of protein synthesis is commonly dysregulated in cancer, most frequently by mutational activation of the phosphoinositide 3-kinase, protein kinase B/Akt/mammalian target of rapamycin (PI3K/Akt/mTOR) pathway. The PI3K/Akt/mTOR pathway promotes cell survival and growth, by inducing the phosphorylation of the small (40S) ribosomal subunit protein S6 (RPS6) and the eukaryotic initiation factor 4e binding protein 1 (4eBP1). These events stimulate polysome assembly and increased cap-dependent (eIF4E-dependent) translation of tumorigenic mRNAs.

Other pathways in addition to the PI3K/Akt/mTOR can cause translational dysregulation in cancer. For example, the rRNA methyltransferase WBSCR22 is involved in the biogenesis of the 40S ribosomal subunit and is overexpressed in invasive breast cancer. The large ribosomal subunit protein 24 (RPL24) is another translation factor previously linked to tumorigenesis. Homozygous RPL24 deficiency is lethal in mice. In contrast, RPL24 haploinsufficient mice are viable with specific eye, skeletal, and coat pigment defects. Interestingly, these RPL24 haploinsufficient mice show greater survival from Akt-induced lymphomagenesis. This protection is associated with an overall decrease in thymocyte protein synthesis. Likewise, RPL24 haploinsufficient mice are protected from Myc-driven tumorigenesis. Myc-induced tumorigenesis arises by increased cap-dependent translation that is also prevented by RPL24 haploinsufficiency. In studies of human lung adenocarcinoma cells depleted of RPL24 by RNA interference, and in RPL24 haploinsufficient mouse embryonic fibroblasts (MEFs), RPL24 reduction is associated with increased p53 expression, indicating that the prevention of tumorigenesis by reduced RPL24 may also depend on a p53-dependent checkpoint mechanism.

A full understanding of the role of RPL24 in tumorigenesis requires mechanistic elucidation of how RPL24 interacts with other ribosomal proteins and translation factors. RPL24 is one of the later proteins to be incorporated into the large ribosomal subunit, where it then regulates the joining of the 60S subunit to the small 40S subunit. Crystallography of the Tetrahymena thermophilis 60S ribosomal subunit and cryo-electron microscopy reconstruction of the Saccharomyces cerevisiae 60S indicate that RPL24 resides on a surface of the 60S ribosomal subunit close to where the eukaryotic initiation factor 6 (eIF6) contacts the 60S. The anti-assembly factor, eIF6, binds to the pre-60S ribosomal subunit and prevents premature association of 60S with the 40S subunit. Following 60S maturation, eIF6 is released, allowing for the joining of the 40S sand 60S subunits to form the 80S ribosome and further assembly of polysomes.

Analyzing a public dataset of RNA profiles reported from 43 pairs of breast cancer and normal breast samples it was observed that most human breast cancers overexpress RPL24 relative to normal breast tissue. As shown herein, RPL24 depletion in breast cancer cells reduces their growth and viability in association with selectively impaired expression of cap-dependent proteins needed for survival and proliferation, while also inhibiting 80S ribosome and polysome assembly by preventing eIF6 release from the 60S subunit. Herein it is also shown that 2-24 h treatment with a pan-inhibitor of class I and II histone deacetylases, trichostatin-A, mimics the above effects of RPL24 depletion, inducing 60S subunit-associated acetylation of RPL24 at K27. TSA also induces acetylation of polysomal RPL24 at K93 and 14 other ribosomal proteins. Comparison of pan-, class-, and isotype-selective histone deacetylase inhibitors indicates that HDAC6 controls total acetylation levels of ribosomal proteins, a conclusion supported by HDAC6 knockdown.

Accordingly, provided herein are methods for treating RPL24-overexpressing cancer in a subject in need thereof, comprising administering to the subject a therapeutically effective amount of an HDAC inhibitor.

The subject considered herein is typically a human. However, the subject can be any mammal for which treatment is desired. Thus, the methods described herein can be applied to both human and veterinary applications.

As such, in one embodiment, provided herein is a method for treating RPL24-overexpressing cancer in a subject in need thereof, comprising administering to the subject a therapeutically effective amount of a compound of formula IV, or pharmaceutically acceptable salts thereof.

In another embodiment is a method for treating RPL24-overexpressing cancer in a subject in need thereof comprising administering to the subject a therapeutically effective amount of Compound 101, or pharmaceutically acceptable salts thereof.

In another embodiment is a method for treating RPL24-overexpressing lung cancer in a subject in need thereof comprising administering to the subject a therapeutically effective amount of a compound of formula IV, or pharmaceutically acceptable salts thereof.

In another embodiment is a method for treating RPL24-overexpressing breast cancer in a subject in need thereof comprising administering to the subject a therapeutically effective amount of a compound of formula IV, or pharmaceutically acceptable salts thereof.

In another embodiment is a method for treating RPL24-overexpressing basal-like breast cancer in a subject in need thereof comprising administering to the subject a therapeutically effective amount of a compound of formula IV, or pharmaceutically acceptable salts thereof.

In another embodiment is a method for treating RPL24-overexpressing Myc-induced cancer in a subject in need thereof comprising administering to the subject a therapeutically effective amount of a compound of formula IV, or pharmaceutically acceptable salts thereof.

In another embodiment is a method for treating RPL24-overexpressing Akt-induced cancer in a subject in need thereof comprising administering to the subject a therapeutically effective amount of a compound of formula IV, or pharmaceutically acceptable salts thereof.

Provided herein are methods for inhibiting migration or invasion, or both, of RPL24-overexpressing cancer cells. In particular, provided herein are methods for inhibiting migration or invasion, or both, of RPL24-overexpressing cancer cells in a subject in need thereof. Specifically, provided herein are methods for inhibiting migration or invasion, or both, of RPL24-overexpressing cancer cells in a subject in need thereof comprising administering to the subject a therapeutically effective amount of an HDAC inhibitor of formula IV.

In an embodiment of any one of the methods provided herein, the HDAC inhibitor is an HDAC-selective inhibitor. In an embodiment, the HDAC-selective inhibitor is HDAC1-selective, HDAC2-selective, HDAC3-selective, or HDAC8-selective. In another embodiment, the HDAC-selective inhibitor is HDAC4-selective, HDAC5-selective, HDAC6-selective, HDAC7-selective, HDAC9-selective, or HDAC10-selective. In another embodiment, the HDAC-selective inhibitor is HDAC11-selective. In another embodiment, the HDAC-selective inhibitor is HDAC6-selective.

### Kits

In other embodiments, kits are provided. Kits provided herein include package(s) comprising compounds or compositions provided herein. In some embodiments, kits comprise an HDAC inhibitor, or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions provided herein to a patient. Kits also can comprise instructions for approved uses of compounds herein by regulatory agencies, such as the United States Food and Drug Administration. Kits can also contain labeling or product inserts for the compounds. The package(s) or any product insert(s), or both, may themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

### DEFINITIONS

Listed below are definitions of various terms used herein. These definitions apply to the terms as they are used throughout this specification and claims, unless otherwise limited in specific instances, either individually or as part of a larger group.

The term “alkyl,” as used herein, refers to saturated, straight- or branched-chain hydrocarbon moieties containing, in certain embodiments, between one and six, or one and eight carbon atoms, respectively. Examples of C₁-C₆ alkyl moieties include, but are not limited to, methyl, ethyl, propyl, isopropyl, n-butyl, tert-butyl, neopentyl, n-hexyl moieties; and examples of C₁-C₈ alkyl moieties include, but are not limited to, methyl, ethyl, propyl, isopropyl, n-butyl, tert-butyl, neopentyl, n-hexyl, heptyl, and octyl moieties.

The number of carbon atoms in a hydrocarbyl substituent can be indicated by the prefix “Cₓ-C_{y},” where x is the minimum and y is the maximum number of carbon atoms in the substituent. Likewise, a C, chain means a hydrocarbyl chain containing x carbon atoms.

The term “alkoxy” refers to an —O-alkyl moiety.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like. In some embodiments, aryl groups have 6 carbon atoms. In some embodiments, aryl groups have from 6 to 10 carbon atoms. In some embodiments, aryl groups have from 6 to 16 carbon atoms. The term “aralkyl,” or “arylalkyl,” as used herein, refers to an alkyl residue attached to an aryl ring. Examples include, but are not limited to, benzyl, phenethyl and the like.

The term “carbocyclic,” as used herein, denotes a monovalent group derived from a monocyclic or polycyclic saturated, partially unsatured, or fully unsaturated carbocyclic ring compound. Examples of carbocyclic groups include groups found in the cycloalkyl definition and aryl definition.

The term “cycloalkyl,” as used herein, denotes a monovalent group derived from a monocyclic or polycyclic saturated or partially unsatured carbocyclic ring compound. Examples of C₃-C₈-cycloalkyl include, but not limited to, cyclopropyl, cyclobutyl, cyclopentyl, cyclohexyl, cycloheptyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl. Also contemplated are monovalent groups derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heterocycloalkyl,” as used herein, refers to a non-aromatic 3-, 4-, 5-, 6- or 7-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The terms “hal,” “halo” and “halogen,” as used herein, refer to an atom selected from fluorine, chlorine, bromine and iodine.

The term “haloalkyl,” as used herein, refers to an alkyl moiety substituted with one or more atoms selected from fluorine, chlorine, bromine and iodine.

The term “haloaryl,” as used herein, refers to an aryl moiety substituted with one or more atoms selected from fluorine, chlorine, bromine and iodine.

The term “pharmaceutically acceptable salt” refers to those salts of the compounds formed by the processes provided herein which are, within the scope of sound medical judgment, suitable for use in contact with the tissues of humans and lower animals without undue toxicity, irritation, allergic response and the like, and are commensurate with a reasonable benefit/risk ratio. Additionally, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts provided herein include the conventional non-toxic salts of the parent compound formed, for example, from non-toxic inorganic or organic acids. The pharmaceutically acceptable salts provided herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

A subject can also refer to an animal model of an RPL24-overexpressing cancer.

The terms “treating” or “treatment” indicates that the method has, at the least, mitigated abnormal cellular proliferation. For example, the method can reduce the rate of RPL24-overexpressing cancer growth in a patient, or prevent the continued growth or spread of the RPL24-overexpressing cancer, or even reduce the overall reach of the RPL24-overexpressing cancer. In another embodiment, the terms “treating” or “treatment” can refer to any improvement in one or more clinical symptoms of an RPL24-overexpressing cancer.

The terms “isolated” or “purified” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

As used herein, RPL24-overexpression refers to the expression, at a level higher than normal expression levels, of the 60S ribosomal protein L24.

As used herein, Akt-induced refers to a state that is triggered by the action of the protein kinase Akt (also known as protein kinase B).

As used herein, Myc-induced refers to a state that is triggered by the action of the transcription factor Myc.

### EXAMPLES

### Example 1

### RPL24 Expression is Transcriptionally Upregulated During Human Breast Tumorigenesis

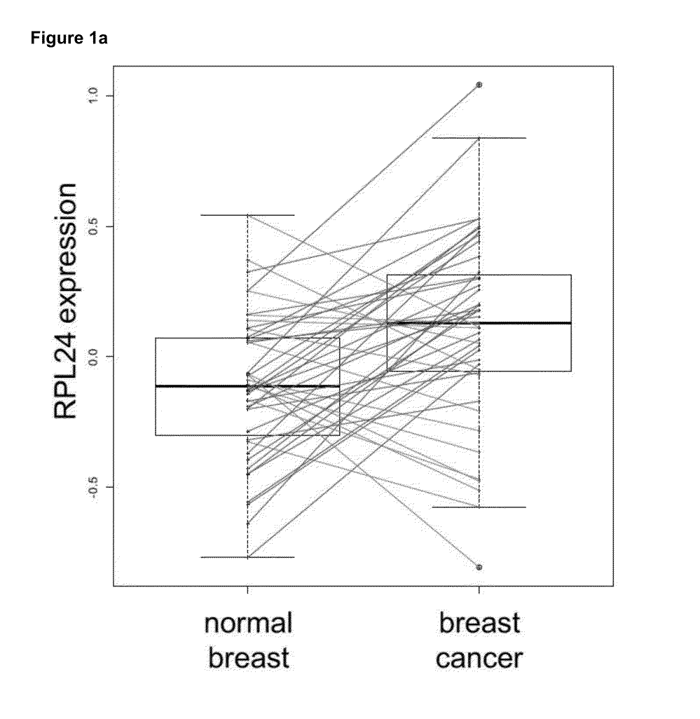

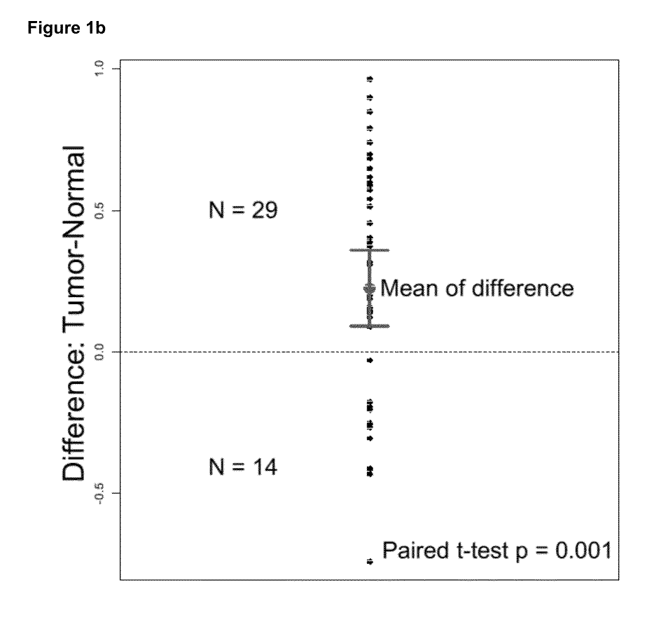

Since RPL24 haploinsufficiency impairs the formation of both Akt-driven and Myc-driven murine malignancies, evidence that RPL24 upregulation may contribute to human tumorigenesis was sought as well. To that end, microarrayed samples of human cancers paired with their normal organ tissue samples were compared. Using a public dataset of RNA profiles reported from 43 pairs of breast cancer and normal breast samples, it was determined that approximately two-thirds of the breast cancers showed increased RPL24 transcript levels relative to their matched normal breast sample (FIG. 1a). The entire group of tumor samples exhibited a significant 20% mean overall increase in RPL24 expression levels (p=0.001), indicating that transcriptional upregulation of RPL24 commonly occurs in human breast tumorigenesis (FIG. 1b).

### Example 2

### RPL24 Knockdown Reduces Breast Cancer Cell Viability while Inhibiting Cap (eIF4eE)-Dependent Expression of Proliferation, Survival and Genome Stability Proteins

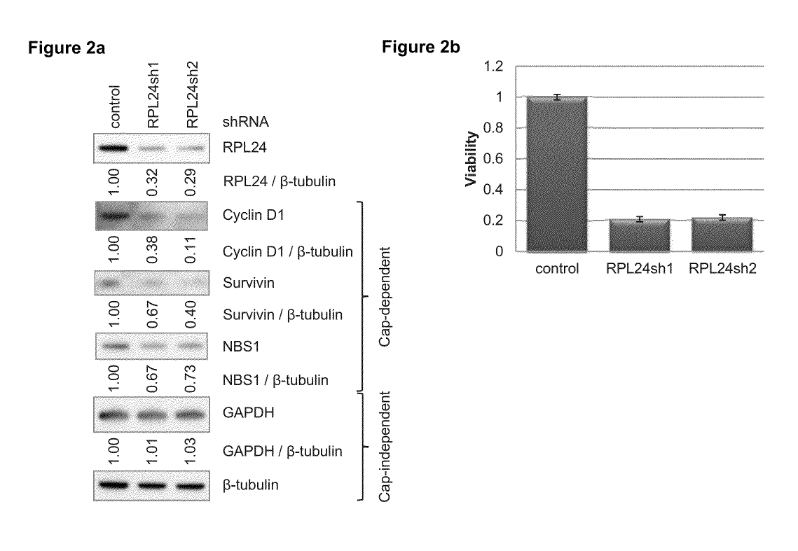

Studies of RPL24 haploinsufficient mice protected from Myc-driven tumorigenesis revealed that dysregulated cap-dependent protein synthesis not only induces tumor formation but also results in cell cycle dysregulation and genomic instability. Since the translation-dependent checkpoint mechanism remains undefined, the impact of RPL24 depletion in a model human breast cancer cell line, SKBR3, sensitive to eIF4E-regulated and cap-dependent translation inhibition was evaluated. Two different RPL24-directed shRNA-expressing lentiviruses were used to decrease RPL24 protein expression by approximately 70% (FIG. 2a). This resulted in a 5-fold (80%) reduction in SKBR3 cell viability measured after 4 days in culture (FIG. 2b). Associated with RPL24 depletion and growth inhibition was a marked reduction in the expression of three different eIF4E-regulated and cap-dependent transcripts necessary for cell proliferation (75% reduction in cyclin D1), survival (46% reduction in survivin), and DNA repair and integrity (30% reduction in NBS1). Protein levels of two housekeeping genes not regulated by eIF4E, GAPDH and β-tubulin, were not affected by RPL24 depletion (FIG. 2a).

### Example 3

### RPL24 Knockdown Reduces 80S and Polysome Assembly while Increasing 60S Retention of eIF6

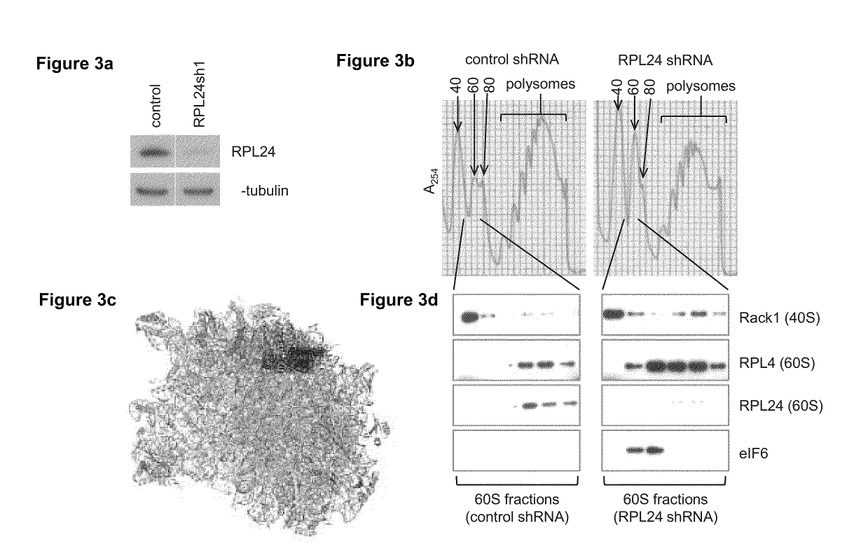

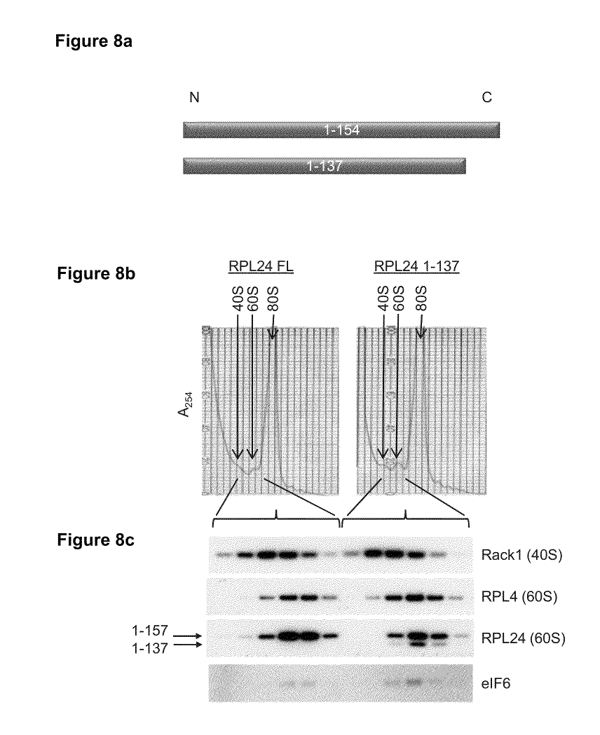

Since RPL24 depletion decreased the levels of three cap-dependently translated proteins, the impact of RPL24 knockdown on overall ribosome and polysome formation in these cells was evaluated. Polysome profiling, which utilizes continuous sucrose gradient fractionation to separate free 40S and 60S ribosomal subunits, 80S ribosomes, and polysomes (two or more ribosomes on one mRNA) was used. The ratio of both 80S ribosomes and polysome peaks relative to free 40S and 60S ribosomal subunits was significantly reduced in SKBR3 cells following efficient RPL24 knockdown (FIG. 3a,b). This observed increase in 40S and 60S subunits relative to 80S ribosomes implies a defect in 40S-60S joining induced by the RPL24 knockdown. Since eIF6 bound to the pre-60S subunit prevents joining of the 40S and 60S subunits and occurs adjacent to RPL24 on 60S (FIG. 3c), immunoblotting on all 60S-containing polysome fractions to evaluate the impact of RPL24 knockdown on eIF6 retention was performed. Probing fractions corresponding to the area of the polysome profile near the 60S peak for Rack1, an obligatory 40S component, confirmed the location of any 40S fractions relative to all 60S fractions, detected by RPL4 (another 60S subunit protein) probing, which also showed the expected 60S loss of RPL24 in the SKBR3 cells expressing RPL24 shRNA. Associated with the observed 60S loss of RPL24 was a striking increase in 60S-associated eIF6 (FIG. 3d). To rule out the possibility that the observed 60S retention of eIF6 might be a false-positive or non-specific artifact of lentiviral expressed RPL24 shRNA, a functionally deficient truncation mutant of RPL24 that eliminates the last 17 amino acids was overexpressed. Polysome profiling of 293T cells expressing intact versus truncated RPL24 protein confirmed that truncated RPL24 can induce 60S retention of eIF6 (FIG. 8).

### Example 4

### Ribosomal Protein Acetylation is Induced by Histone Deacetylase Inhibition

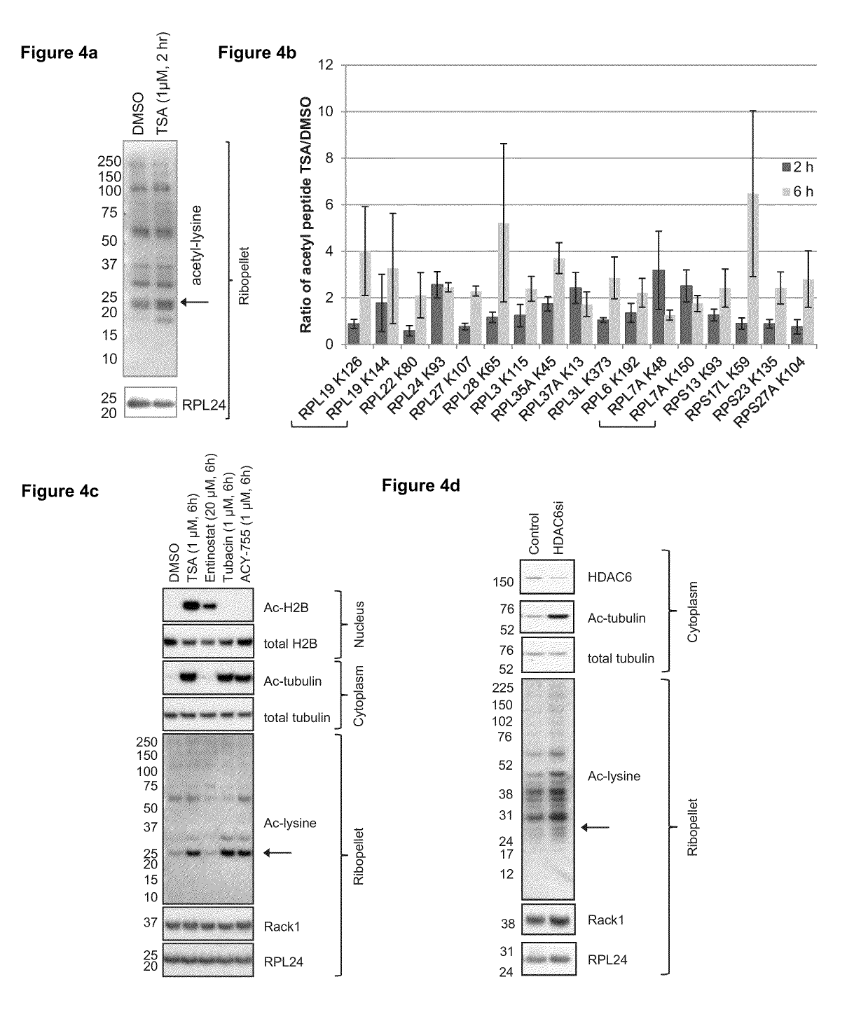

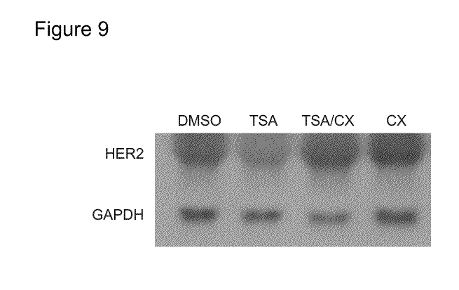

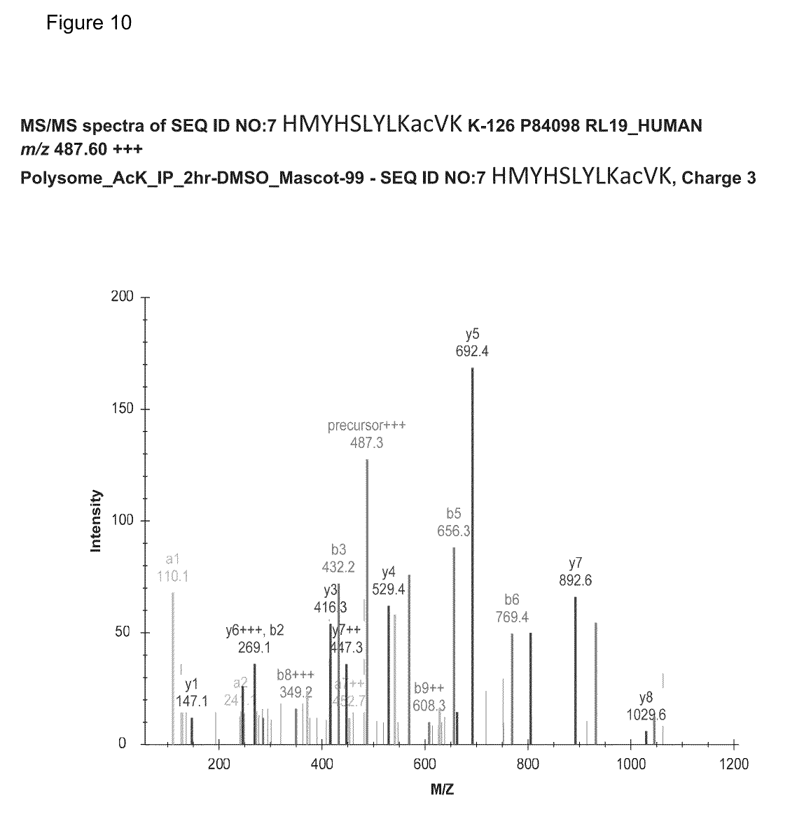

Previous studies have shown that pan-inhibitors of class I and II histone deacetylases, like TSA, can rapidly destabilize a number of oncogenic transcripts including HER2 in a cycloheximide-dependent manner (FIG. 9). Since cycloheximide inhibits polysome formation, these results indicated that polysomes are involved in HER2 mRNA decay. Thus, SKBR3 cells were treated with TSA to evaluate polysome protein acetylation and determine if, similar to RPL24 depletion, histone deacetylase inhibition can affect ribosome assembly dynamics. To detect early (2 h) ribosome or polysome acetylation following histone deacetylase inhibition treatment (1 μM TSA), SKBR3 polysomes were isolated using a discontinuous sucrose gradient as previously described. Western blots using an antibody against acetylated lysine residues showed several TSA-induced bands, including a prominent TSA-induced acetyl-lysine protein band co-migrating with RPL24 (FIG. 4a, indicated by arrow), while total RPL24 levels were not altered by TSA. Mass spectrometry studies indicate that 15 ribosomal proteins, 11 large subunit proteins (RPL24 included) and 4 small subunit proteins, underwent at least a two-fold induction in acetylation following 2 h or 6 h TSA treatment (1 μM) (FIG. 4b, FIG. 10a-q).

Like TSA, the HDAC6 (class IIb)-selective inhibitors, Tubacin and Compound 101, as well as HDAC6 siRNA, all induce tubulin acetylation as expected as well as ribosomal protein acetylation, including the band that co-migrates with RPL24 (FIG. 4c,d, indicated by arrows). Although the class I-specific histone deacetylase inhibitor, Entinostat, induces histone H2B acetylation without acetylating tubulin, it does not alter ribosomal protein acetylation even at a dose of 20 μM (FIG. 4c). Thus, the tubulin acetylating effects of pan-histone deacetylase inhibition, known to be mediated by inhibition or knockdown of HDAC6, correspond to the observed ribosome and RPL24 acetylation responses induced by TSA.

### Example 5

### Like RPL24 Knockdown, Histone Deacetylase Inhibition Reduces 80S Assembly While Increasing 60S Retention of eIF6 and Reduces Expression of Cap (eIF4E)-Dependently Translated Proteins

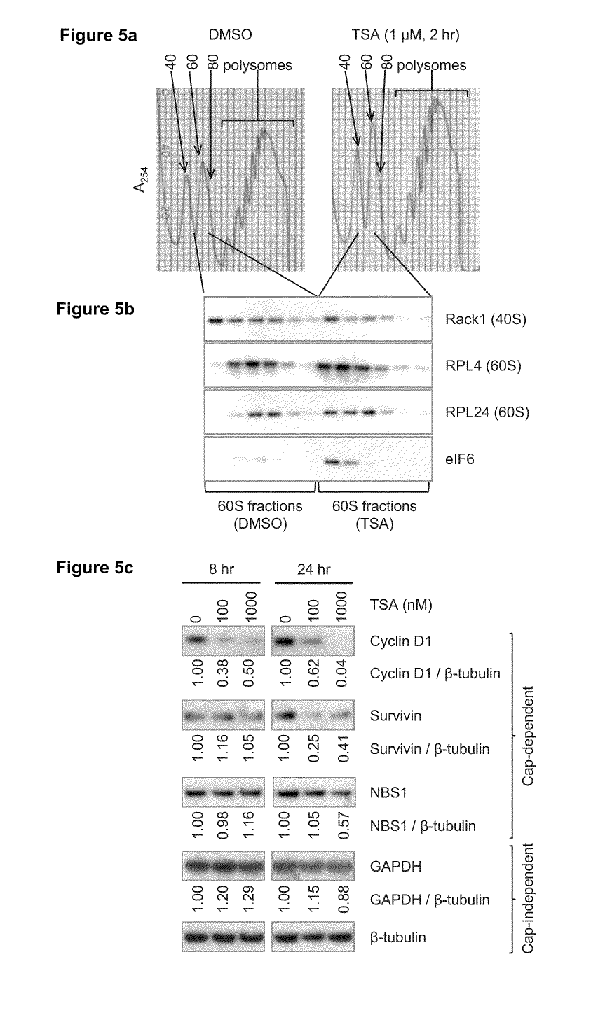

Using continuous sucrose gradient fractionation of SKBR3 polysomes, 2 h culture treatment with TSA reduced 80S and polysome assembly (FIG. 5a) while increasing 60S retention of eIF6 without reducing 60S RPL24 levels (FIG. 5b). This result is comparable to that produced by RPL24 knockdown (FIG. 3) or truncation (FIG. 8). Furthermore, similar to RPL24 knockdown, 24 h TSA treatment reduced the expression of the cap-dependently translated proteins cyclin D1, survivin, and NBS1 relative to the housekeeping proteins GAPDH and β-tubulin (FIG. 5c). Shorter (8 h) TSA treatment reduced cyclin D1 levels but not survivin or NBS1 levels. The more rapid reduction of cyclin D1 levels was likely caused by the known effects of TSA on cyclin D1 transcription and transcript stability in addition to its effects on translation.

### Example 6

### Histone Deacetylase Inhibition Enhances Lysine (K27) Acetylation on 60S RPL24

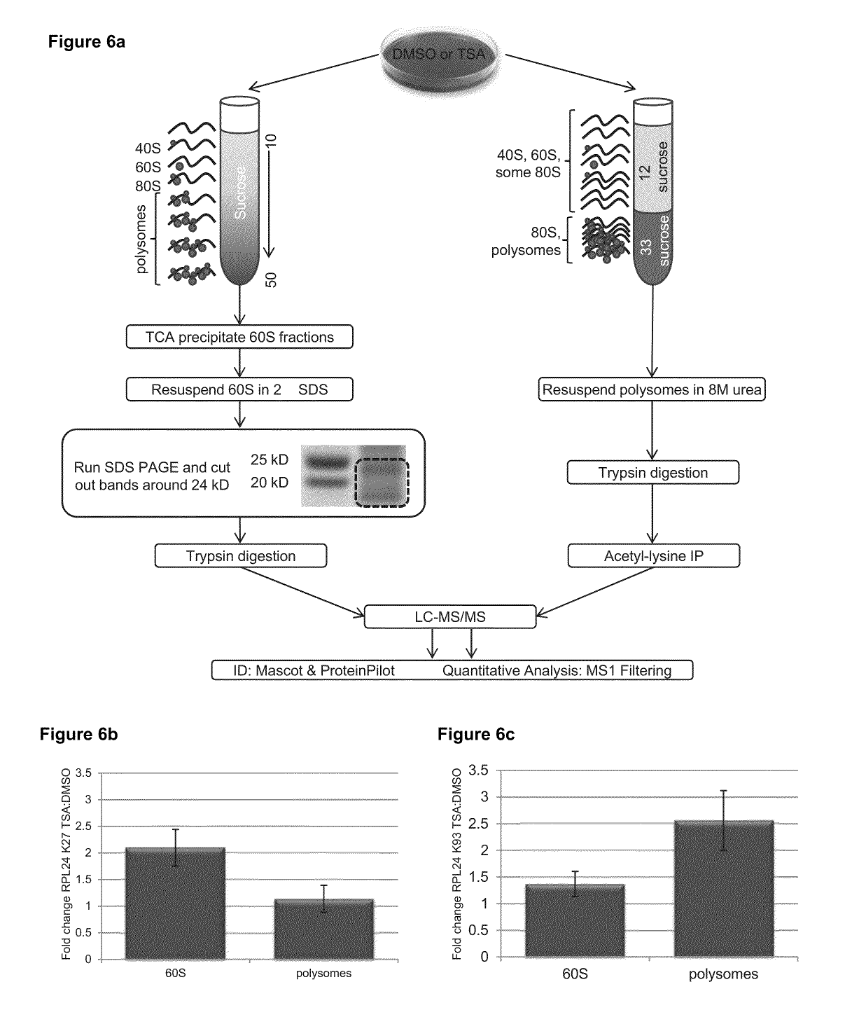

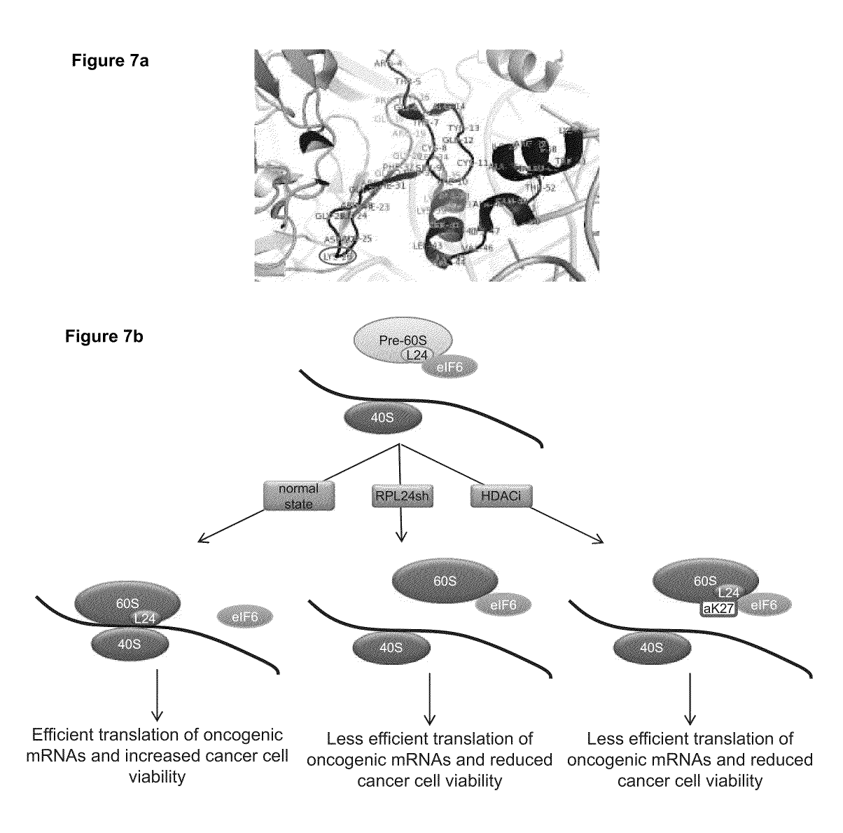

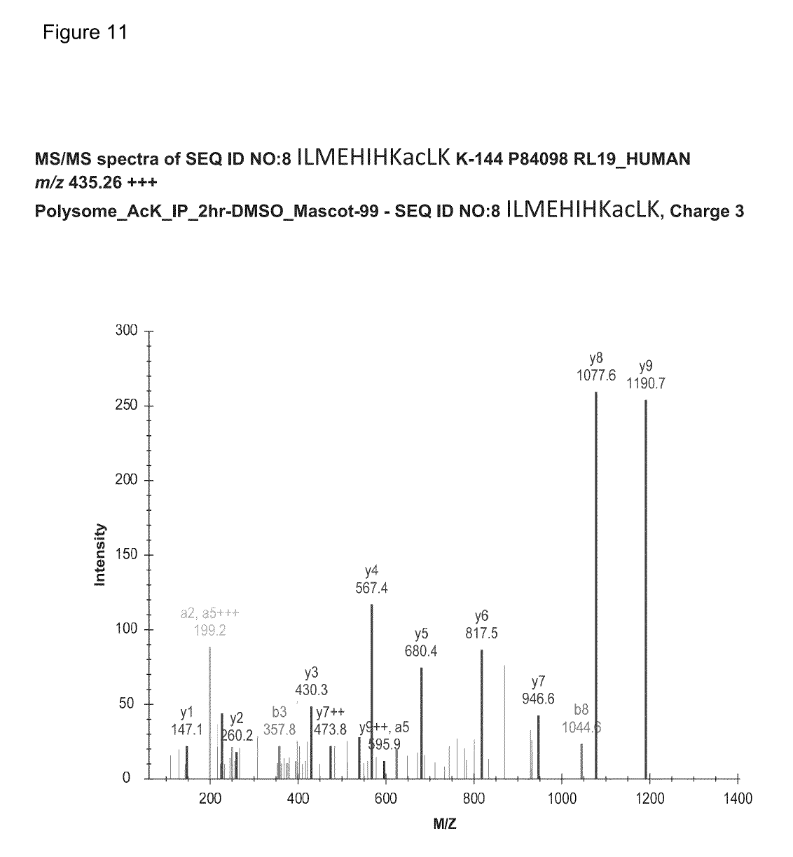

Mass spectrometry studies were performed to identify sites of lysine (K) acetylation within RPL24 induced by histone deacetylase inhibition. Continuous and discontinuous sucrose gradient fractionations were performed to isolate 60S subunits and total polysomes respectively. Polyacrylamide gel electrophoresis was then performed on 60S and polysome samples and RPL24-containing bands were excised, trypsin digested, and subjected to mass spectrometry (LC-MS/MS) (FIG. 6a). Among several detected acetylated RPL24 peptides, two were increased by TSA treatment; TDGKacVFQFLNAK (acetyl-K27) (SEQ ID NO:1) and AITGASLADIM*AKacR (acetyl-K93) (SEQ ID NO:2), where the internal lysines in both peptides are N-acetylated (Kac). As the MS experiments of the 60S polysome were performed after in-gel digestion the methionine residue of the second peptide was predominantly oxidized (M*=M+16), as commonly observed during SDS PAGE processing. In independent, in-solution digestion experiments, the corresponding non-oxidized form of acetylated peptide AITGASLADIMAKacR (SEQ ID NO:3) with correlating MS/MS fragmentation pattern was identified. Representative spectra are shown for TDGKacVFQFLNAK (acetyl-K27) (SEQ ID NO:1) and AITGASLADIM*AKacR (acetyl-K93) (SEQ ID NO:2)) (FIG. 11a,b). In 3 biological replicate experiments, the amount of K27-acetylated RPL24, normalized to total protein concentration within the 60S subunit, was increased at least 2-fold within 2 h of TSA treatment. However, there was no significant induction of RPL24 K27 acetylation found within polysomes (not containing 60S subunits) (FIG. 6b). In contrast, RPL24 K93 acetylation within the 60S subunits was not significantly changed by TSA treatment, yet K93 acetylation was induced 2.5-fold within RPL24 associated with polysomes (FIG. 6c). Given the proximity of the T. thermophilia RPL24 K26 site (that resides in a homologous region to human K27) to eIF6 (FIG. 7a), these findings implicate involvement of the TSA induced acetylation of RPL24 at K27 in preventing 40S-60S subunit joining and 60S retention of eIF6.

### Example 7

### Comparison of RPL24 Transcript Levels in Matched Breast Carcinoma and Normal Tissue

RPL24 transcript levels from a public dataset of matched human breast cancers and normal mammary tissue are compared. RPL24 is shRNA-depleted in SKBR3 human breast cancer cells and the effects of this knockdown on cell viability, expression of growth and survival-promoting proteins relative to housekeeping proteins, and changes in ribosomal proteins and their polysome assembly are evaluated. These RPL24 knockdown effects are compared to SKBR3 treatment responses following pan-, class-, or isotype-selective histone deacetylase inhibition, whose selective abilities to acetylate RPL24 and other ribosomal proteins are assessed by immunoblotting and mass spectrometry.

### Example 8

### Analysis of RPL24 Expression in Patient-Matched Breast Carcinoma and Normal Breast Tissue

Expression data from 43 patient-matched breast carcinoma and normal breast tissue samples assayed on Affymetrix U133A microarrays (GSE15852) is obtained from the Gene Expression Omnibus (GEO). Raw data is RMA-normalized, annotated using its associated platform annotation file (GPL96-39578) and mean-centered. Expression levels of the RPL24 probe within the patient-matched tumor and normal samples are obtained and compared. Significance is assessed using the paired t-test.

### Example 9

### Cell Culture

SKBR3 human breast cancer cells (American Type Culture Collection (ATCC), Rockville, Md.) are grown in McCoy's 5A media supplemented with 10% fetal bovine serum (FBS) and L-glutamate. 293T cells (American Type Culture Collection (ATCC), Rockville, Md.) are grown in DMEM with 10% FBS and L-glutamate.

### Example 10

### shRNA and Retroviral Infection

Lentiviral vectors containing shRNAs toward RPL24, TRCN0000117642/RPL24sh1/target sequence CCTGAAGTTAGAAAGGCTCAA (SEQ ID NO:4) and TRCN0000117643/RPL24sh2/target sequence GTGCATCTCTTGCTGATATAA (SEQ ID NO:5), and a green fluorescent protein control RHS4459/target sequence TACAACAGCCACAACGTCTAT (SEQ ID NO:6) are purchased from Thermo Scientific (formerly Open Bio systems, Cincinnati, Ohio). shRNA expressing lentiviruses are produced as previously described. Briefly, 293T cells are transfected with lentiviral vectors along with packaging vectors. One day later, media is changed to Optimem (Life Technologies, Grand Island, N.Y.) and the virus is collected for two days and concentrated as outlined previously. SKBR3 cells are infected in the presence of 6 μg/ml polybrene with a multiplicity of infection of −2. One day after infection media is changed to regular growth media in the case of transient infections or growth media with 0.5 μg/ml puromycin in the case of stable transfections.

### Example 11

### siRNA Transfection

The following siRNAs are purchased from (Thermo Scientific-Dharmacon, Chicago, Ill.): HDAC6-targeting smart pool (L-003499-00) and non-targeting control pool (D-001810-10-05). Lipofectamine 2000 (Life Technologies) is used to transfect SKBR3 cells per manufacturer's protocol. Cells are analyzed 72 hours after transfection.

### Example 12

### Viability Assay

Cells infected with different shRNA-expressing lentiviruses are plated in 96-well plates at a density of 5,000 cells per well. Three hours after plating (T₀), a base line viability reading is taken using the CellTiter-Glo Luminescent Cell Viability Assay (Promega, Madison, Wis.). Four days later (T₄) another reading is taken using the same assay. For each treatment, each of three T₄ data points is divided by the average of all three T₀ data points for that treatment. The data from the RPL24 shRNA-treated cells is then normalized to that from the control cells. Data is represented by the mean and standard deviation of triplicates.

### Example 13

### Cell Lysis and Immunoblotting

Cells are lysed in RIPA buffer (10 mM Tris-HCL (pH 8.0), 1 mM EDTA, 0.5 mM EGTA, 1% triton X-100, 0.1% sodium deoxycholate, 0.1% SDS, 140 mM NaCl, 20 mM NaF, Complete EDTA-free protease inhibitor tablets (Roche Diagnostics Corp., Basel, Switzerland) and the phosphatase inhibitor cocktail PhosSTOP (Roche)), the latter two as indicated by the manufacturer's protocol. Equal amounts of protein are diluted in 2× sample buffer. Immunoblots on PVDF (Polyvinyldene Fluoride) membranes are blocked with nonfat milk in tris-buffered saline with 0.05% tween-20 (TBST). The following antibodies are incubated with membranes in 5% nonfat milk in TBST: RPL24 (Proteintech, Chicago, Ill.), Cyclin D1, Rack1, RPL4 (Santa Cruz Biotechnology, Santa Cruz, Calif.), NBS1, GAPDH (EMD Millipore Corporation, Chicago, Ill.), Survivin, β-tubulin, acetyl-lysine, eIF6, acetyl-H2B, H2B, (Cell Signaling Technology, Boston, Mass.), acetyl-tubulin, tubulin (Sigma Aldrich (St. Louis, Mo.))

### Example 14

### Isolation of Ribosomes

Cells, plated at −90% confluency, are treated as indicated. After treatment, cells are treated with 50 μg/ml cycloheximide for 15 minutes. Cells are lysed with a buffer containing 10 mM HEPES, 10 mM KCl, 75 mM NaCl, 10 mM MgCl₂, 0.35% NP40, pH 7.9 supplemented with Complete EDTA-free protease inhibitor tablets, PhosSTOP phosphatase inhibitor tablets (Roche) per manufacturer's instructions, 50 μg/ml cycloheximide, SUPERase RNase inhibitors (Life Technologies) per manufacturer's instructions, 15 μM TSA, and 5 mM nicotinimide to inhibit histone deacetylases. Supernatants are collected as cytoplasmic preparations. Where indicated, pellets containing nuclei are resuspended in RIPA buffer (described above). The suspension is spun at 13,000 rpm for 5 min and supernatants are collected as nuclear preparations.

Ribosomes are subsequently isolated from cytoplasmic preparations as described previously. Briefly, lysates are layered on top of a 12% and 33% discontinuous sucrose gradient and spun at 38,000 rpm for 2 h. The resulting polysome pellet is then resuspended, stripped of RNA with acetic acid, and then pelleted with acetone. The pellet is then resuspended in 8 M urea, 2% CHAPS, and 25 mM dithiothreitol (DTT).

Polysome profiles to separate the 40S, 60S, 80S and polysomes are carried out by layering cell lysates over a continuous 10-50% sucrose gradient and spun at 38,000 rpm for 2 h as previously described. Fractions are collected using a Retriever 500 fraction collector with a UV (UA6) detector (ISCO Teledyne (Lincoln, Nebr.)).

### Example 15

### Visualization of Crystallography Data

Pymol software (Schrodinger, Mannheim, Germany) is used to visualize RPL24 and eIF6 on previously published crystallography data of the Tetrahymena thermophilia 60S ribosomal subunit (human gene names used) bound to eIF6.

### Example 16

### Drugs

TSA is obtained from Sigma Aldrich, Entinostat from Syndax (Waltham, Mass.) and Tubacin from Caymen Chemicals (Ann Arbor, Mich.). Compound 101 is obtained from Acetylon Pharmaceuticals (Boston, Mass.).

### Example 17

### Mass Spectrometry

To prepare polysome samples for mass spectrometry, cells are treated with a histone deacetylase inhibitor and polysome pellets are prepared using a discontinuous sucrose gradient as described above. Protein concentration is determined using the Pierce BCA Protein Assay Kit (Thermo Scientific) and equal amount of protein are trypsin digested. Acetyl-lysine immunoprecipitations are carried out on resultant peptides using an antibody from Cell Signaling Technology. Acetyl-proteins are then eluted, extracted, and desalted.

To determine the acetylation status of 60S subunit proteins, cells are treated with histone deacetylase inhibitor and polysome profiles are performed as described above. The four fractions representing the 60S subunit are identified via western blots for Rack1 and RPL24. Those 60S fractions are then TSA precipitated and reconstituted in 2% SDS. 60S subunit proteins are resolved using 4-12% Bis-Tris gels and stained with Imperial Protein Stain (Thermo). Gel bands are excised, diced into small pieces, destained, reduced with 10 mM dithiothreitol, and alkylated with 5 mM iodoacetamide. In-gel trypsin digestion is performed using a 1:20 enzyme to protein ratio for 16 h at 37° C. Resultant peptides are extracted and desalted.

Three biological replicates of polysome or 60S samples are then analyzed by LC-MS/MS using a quadrupole time-of-flight (QqTOF) TripleTOF 5600 mass spectrometer (AB SCIEX, Dublin, Calif.) coupled to an Eksigent (Dublin, Calif.) nanoLC Ultra, 2D plus. Briefly, the resulting peptides are chromatographically separated on a C-18 reversed-phase analytical column (75 μm I.D.) connected to the TripleTOF 5600 operating in data dependent mode (1 MS1 survey scan followed by 30 MS/MS scans per 1.8 second acquisition cycle). Mascot v2.3.02 and ProteinPilot v4.5 data base searches are employed for peptide identification (Supplemental Table 1a-b) using a false discovery rate analysis (FDR) of 0.01. For MS/MS spectral data of acetylated peptides see Supplemental FIGS. 3 and 4 and further, more detailed interactive viewing of spectral libraries at the Panorama webserver (University of Washington, Seattle), at https://daily.panoramaweb.org/labkey/project/Gibson/Polysomes_Benz2/begin.view?. Quantitative data analysis of acetyl-lysine peptides is performed by integration of selected molecular ion intensities using Skyline MS1 Filtering as previously described. The average signal intensity, as determined by the area under the curve (AUC) of the LC chromatogram, of the replicate biological samples is calculated. The amount of acetylated peptide normalized to total protein loaded onto the gel for each condition is determined and the fold induction upon TSA treatment is then calculated.

### Example 18

### Cloning

Full length (amino acids 1-154) and truncated (amino acids 1-137) RPL24 is PCR amplified from pCMV6-XL5-RPL24 (OriGene, Rockville, Md.) using primers containing EcoR1 and Not1 sites. The amplicons are then cloned into the pCMV6-KanNeo vector (OriGene) using standard cloning techniques.

### Example 19

### Transfection

293T cells (ATCC) are transfected with Lipofecatmine 2000 (Life Technologies) according to the manufacturer's protocols.

### Example 20

### RNA Isolation and Northern Blots

Cells are harvested and RNA is extracted using Trizol (Life Technologies) per manufacturer's protocol. Northern blots are performed as previously described. Briefly, RNA is then electrophoresed into 1% agarose-formaldehyde gels and transferred onto PVDF membranes. Membranes are then hybridized with ³²P-labelled cDNA probes for HER2 or GAPDH, washed, and visualized by autoradiography.

### SUMMARY

Human breast cancers express significantly more RPL24 than matched normal samples. Depletion of RPL24 protein by ≧70% in SKBR3 cells reduced viability by 80% and decreased protein expression of cyclin D1 (75%), survivin (46%) and NBS1 (30%) without altering GAPDH or beta-tubulin levels. RPL24 knockdown reduced 80S subunit levels relative to 40S and 60S levels, and increased 60S retention of the anti-assembly factor eIF6, effects that were mimicked by 2-24 h treatment with a pan-histone deacetylase inhibitor, which induced acetylation of 15 different polysome-associated proteins including RPL24. K27 was identified as the site of PL24 acetylation associated with impaired 60S to 80S maturation. HDAC6-selective inhibition or its knockdown similarly induced ribosomal acetylation. Histone deacetylase inhibitor treatment does not alter RPL24 levels but induces RPL24 K27 acetylation within the 60S subunit, and also mimics the RPL24 depletion effects, the most notable being markedly reduced viability of oncogenic cells. These results demonstrate histone deacetylase inhibition with a compound of formula IV can treat RPL24-overexpressing cancer.

### DESCRIPTION OF DRAWINGS

FIG. 1: RPL24 expression is transcriptionally upregulated during human breast tumorigenesis. RPL24 expression levels were analyzed from the dataset presented in Pathology, research and practice 2010, 206(4):223-228. (a) Box plot of RPL24 expression levels in patient-matched breast carcinoma and normal breast tissues. Lines connect paired data from each patient; and line color reflects relative levels of RPL24 in each paired sample (red: tumor>normal; green: normal>tumor). (b) Differences in RPL24 expression levels between each breast carcinoma and normal breast sample pair. The mean of the differences+SD are shown in red. P-value was obtained using a paired t-test.

FIG. 2: RPL24 knockdown reduces breast cancer cell viability while inhibiting cap (eIF4E)-dependent expression of proliferation, survival and genome stability proteins. SKBR3 cells were infected with lentiviruses expressing a GFP control or RPL24-targeting shRNA. After one week of puromycin selection, cells were plated in 96-well plates for viability assays and lysates were taken in parallel for western blots. (a) Western blots were performed on lysates from an equal number of cells using antibodies toward the indicated proteins. (b) Viability assay readings were taken three hours after plating (day 0) and four days after plating (day 4). The day 4 results were normalized for plating efficiency using the day 0 values. Error bars represent three replicate samples.

FIG. 3: RPL24 knockdown reduces 80S and polysome assembly while increasing 60S retention of eIF6. (a,b,c) SKBR3 cells were infected with lentiviruses expressing a GFP control or RPL24-targeting shRNA for three days. (a) Western blots using the indicated antibodies were performed on total cell lysates to assess knockdown efficiency. (b) Lysates were applied to a continuous sucrose gradient (10-50%) and ultracentrifugation followed by fractionation was performed to separate ribosomal subunits and polysomes. (c) Pymol software was used to visualize the location of RPL24 (blue) relative to eIF6 (green) on the previously published structure of the 60S subunit in complex with eIF6. (d) Western blots using the indicated antibodies were performed on fractions from the 60S peaks using the indicated antibodies.

FIG. 4: Ribosomal protein acetylation is induced by histone deacetylase inhibition. (a-c) SKBR3 cells were treated with the indicated drugs for the indicated period of time. (d) SKBR3 cells were transfected with the indicated siRNAs and allowed to incubate for 72 hours. (a, c, d). The indicated western blots were performed in ribopellets, total cytoplasmic lysates, or nuclear extracts. (b) Mass spectrometry was performed on ribopellets as described in materials and methods and in FIG. 6. The fold change in acetylated peptide to total peptide case by TSA treatment is plotted. Only proteins that underwent at least a two-fold induction upon TSA treatment are shown. Error bars represent the standard error of the mean for three biological replicates.

FIG. 5: Like RPL24 knockdown, histone deacetylase inhibition reduces 80S assembly while increasing 60S retention of eIF6 and reduces expression of cap (eIF4)-dependently translated proteins (a,b) SKBR3 cells were treated with TSA (1 μM, 2 h). (a) Polysome profiles were carried out as previously described. (b) Western blots using the indicated antibodies were performed in fractions representing the 60S subunits. (c) SKBR3 cells were treated with TSA for the indicated doses and times, and proteins were identified by western blotting as indicated.

FIG. 6: Histone deacetylase inhibition enhances lysine (K27) acetylation on 60S, but not polysomal RPL24. (a) Schematic of mass-spectrometry-based techniques to analyze ribosomal protein acetylation. SKBR3 cells were treated with TSA (1 μM, 2 h or 6 h). To isolate 60S subunits, polysome profiles were performed and 60S fractions were TCA precipitated. Concentrated 60S samples were resolved on 4-12% bis-tris gels and RPL24-containing bands were excised and trypsin digested. In parallel, polysomes were isolated using a discontinuous sucrose gradient as described. Trypsin digestions and acetyl lysine immunoprecipitations were subsequently carried out. Mass spectrometry was performed on 60S-associated RPL24-containing gel bands or polysome-containing acetyl-lysine immunoprecipitations as described in the methods section. (b,c) On 60S-associated and polysome-associated RPL24, the fold induction caused by TSA (1 μM, 2 h) of K27 (b) or K93 (c)-acetylated peptide (normalized to total protein concentration) was plotted. Error bars represent the standard error of the mean for three biological replicates. Note: the data for K93 acetylation of RPL24 K93 is also shown in FIG. 4b.

FIG. 7: Schematic for modulation of ribosome assembly by RPL24 acetylation. (a) A magnified portion of the RPL24 (blue)-eIF6 (green) interface, visualized with Pymol software, from previous x-ray crystallography data, is shown (zoomed out view shown in FIG. 3c). T. thermophilia RPL24 residues are labelled and K26, which resides in a region of RPL24 homologous to where human K27 resides, is circled. (b) eIF6 binds to the pre-60S near RPL24 to prevent premature association of the 40S and 60S ribosomal subunits; eIF6 is then released from the mature 60S, allowing it to join with the 40S to form the 80S ribosome. The model indicates that either RPL24 depletion or TSA (histone deacetylase inhibitor)-induced RPL24 acetylation on K27 prevents eIF6 release and 80S formation.

FIG. 8: Expression of truncated RPL24 increases association of eIF6 with 60S fractions in 293T cells. (A-C) 293T cells were transfected with either full length (amino acids 1-154) or truncated RPL24 (amino acids 1-137). (B) Two days later, cells were lysed and polysome profiles were performed. (C) Western blots using antibodies toward the indicated proteins were performed on 60S fractions.

FIG. 9: TSA-induced HER2 mRNA decay is abrogated by cycloheximide treatment. SKBR3 cells were treated with TSA (1 μM, 6 h) and/or cycloheximide (CX, 50 μg/ml, 6 h) or with the respective vehicle controls. RNA was isolated and northern blotted for HER2 and GAPDH transcript levels as shown.

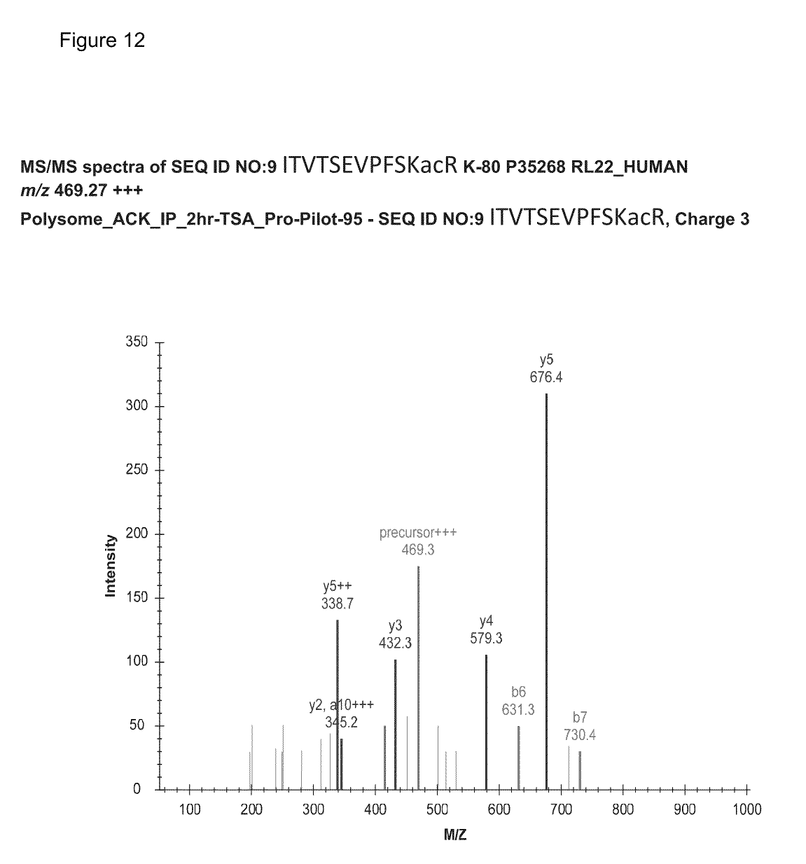

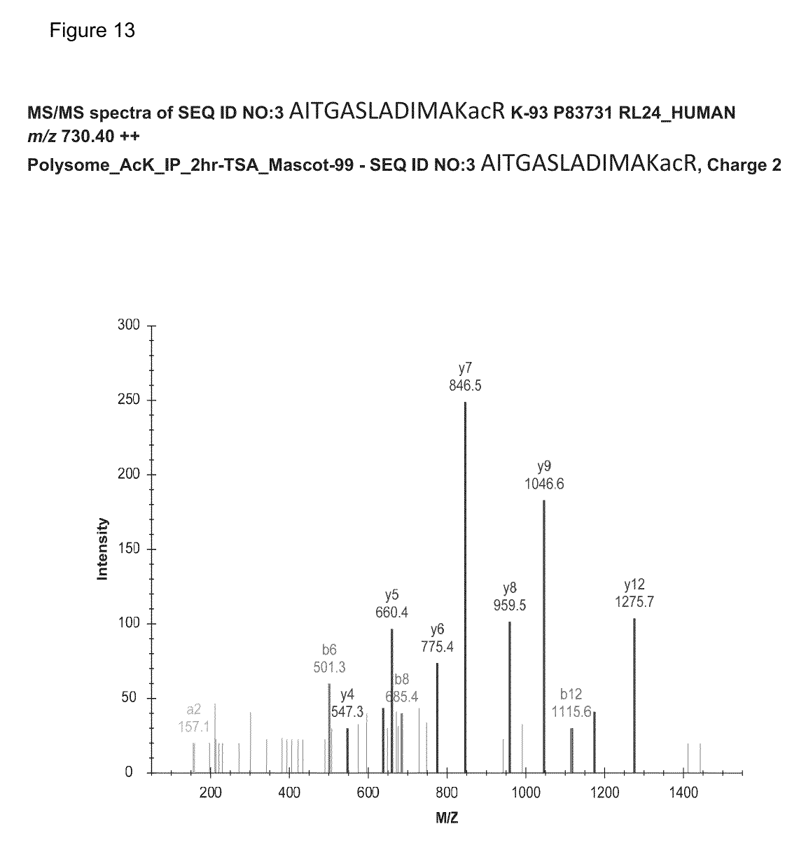

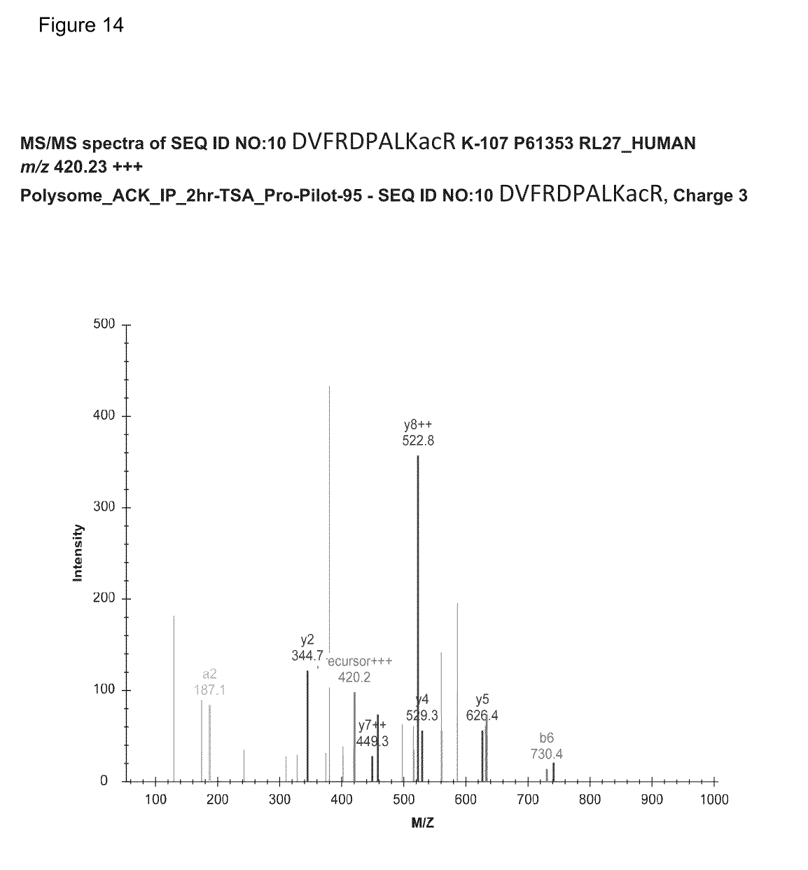

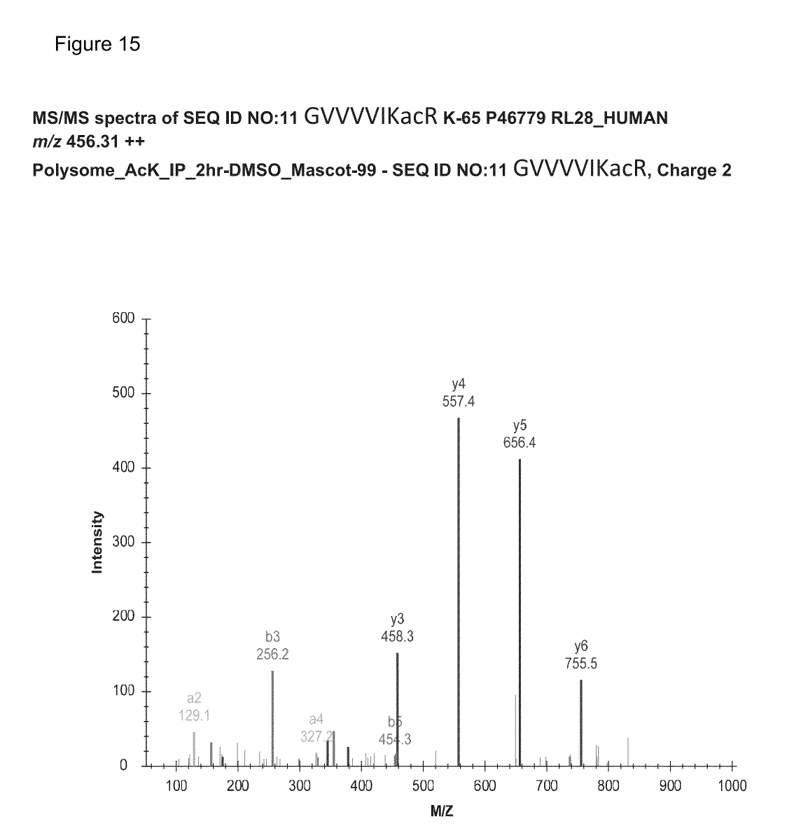

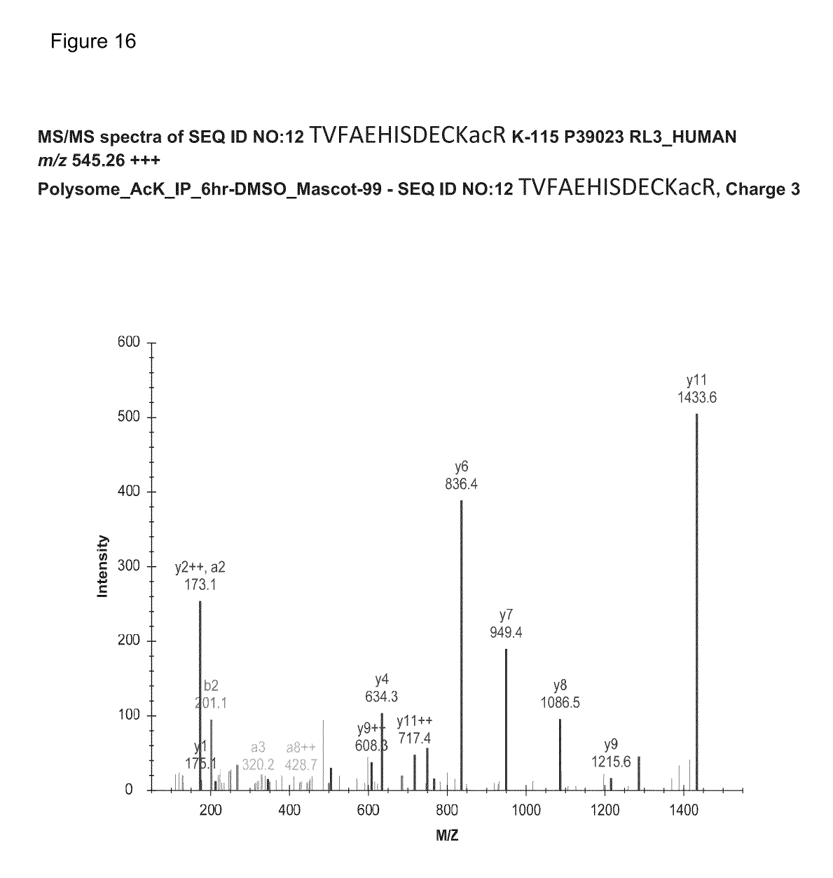

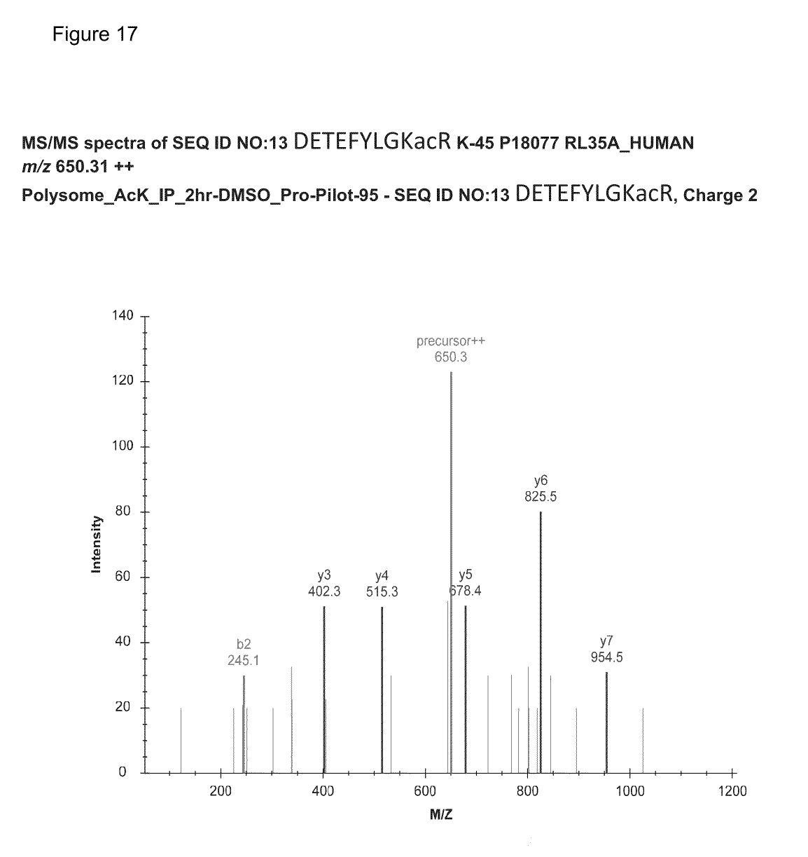

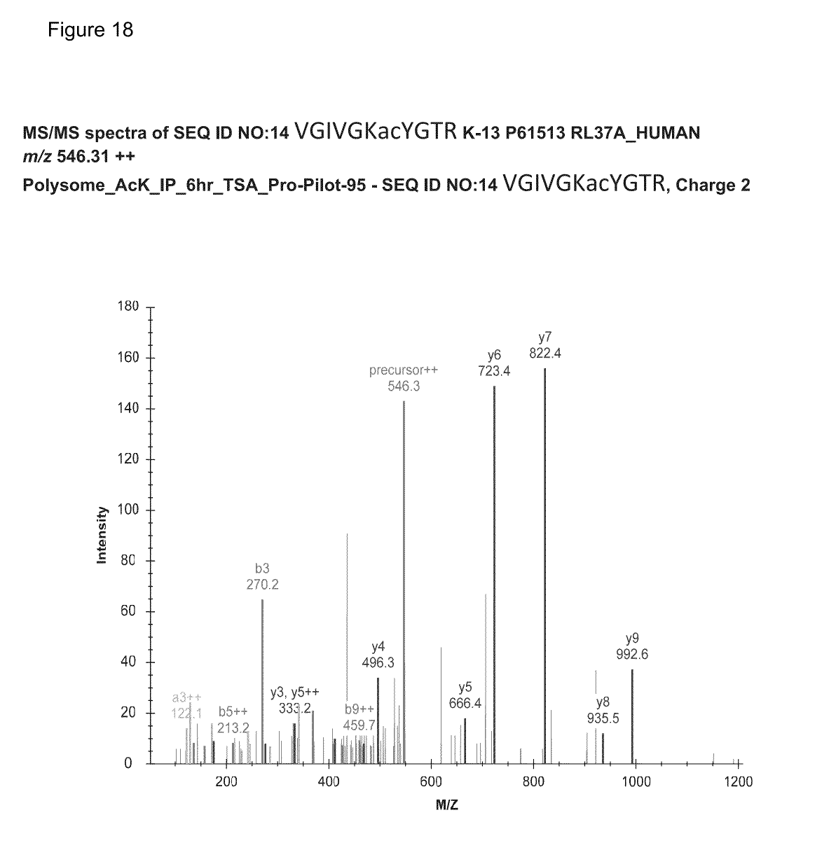

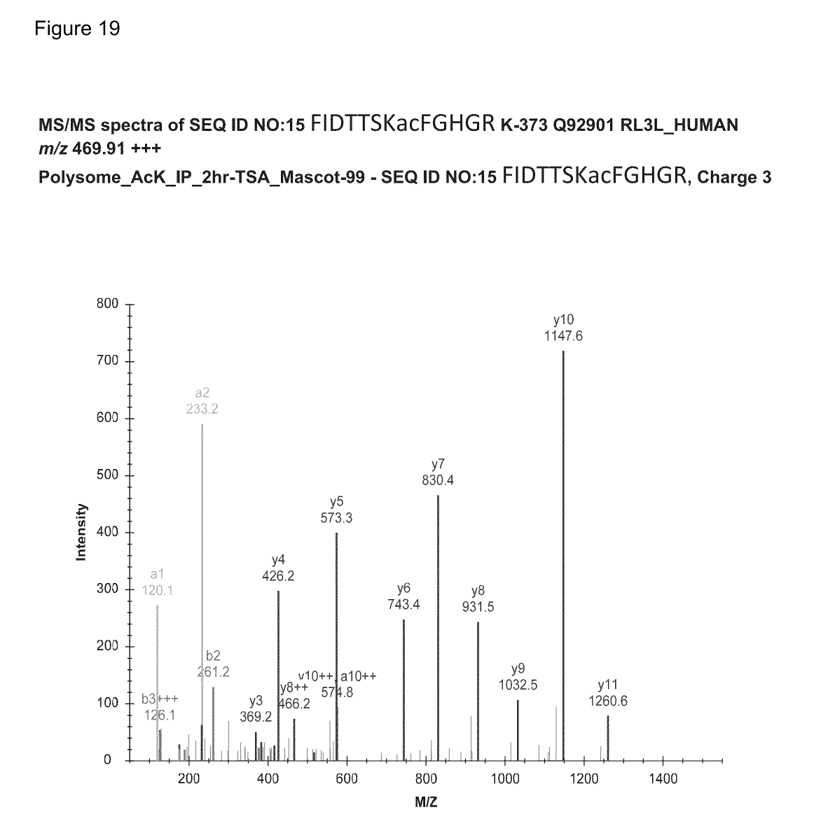

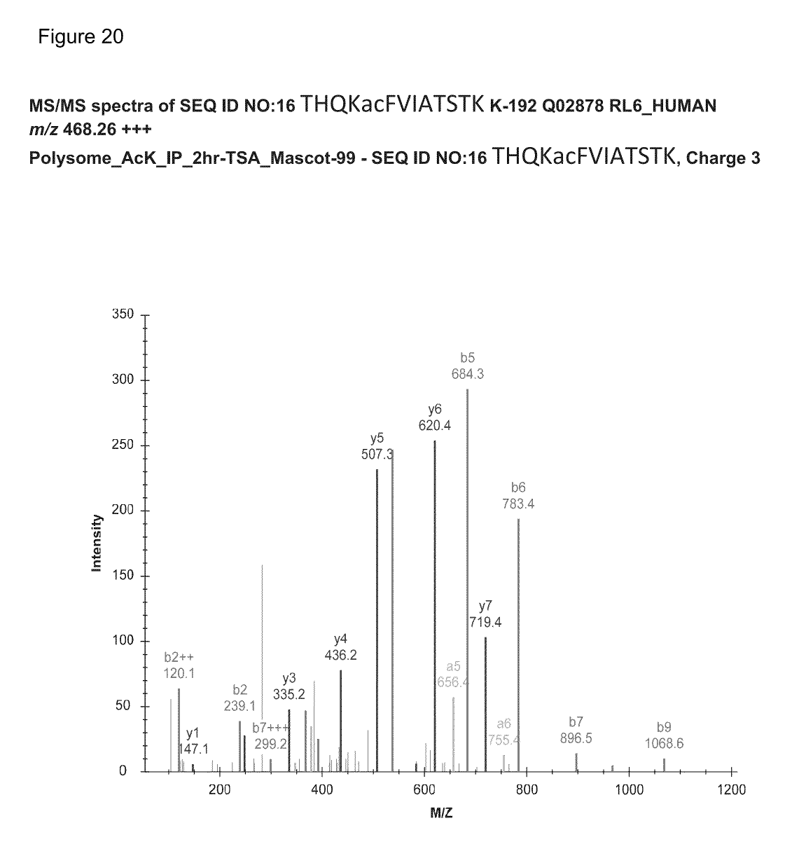

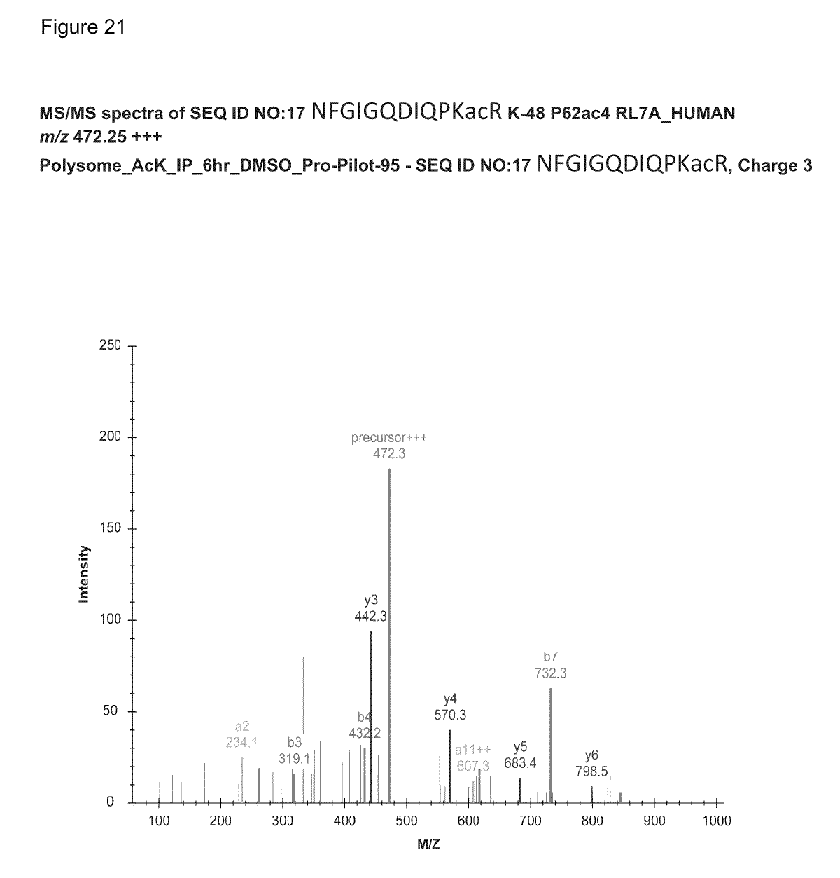

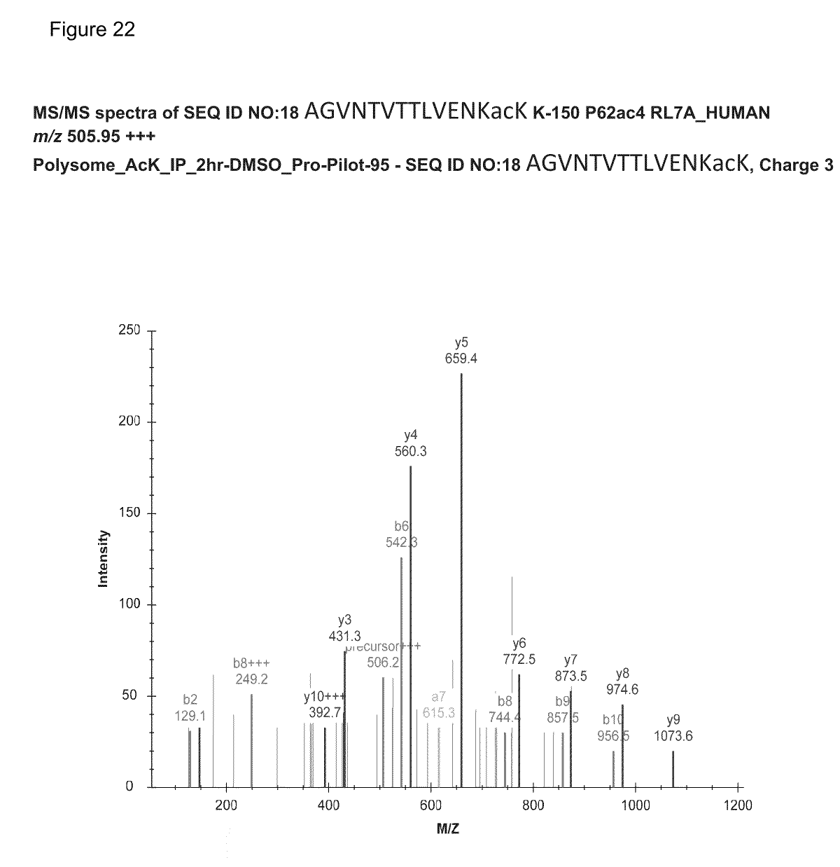

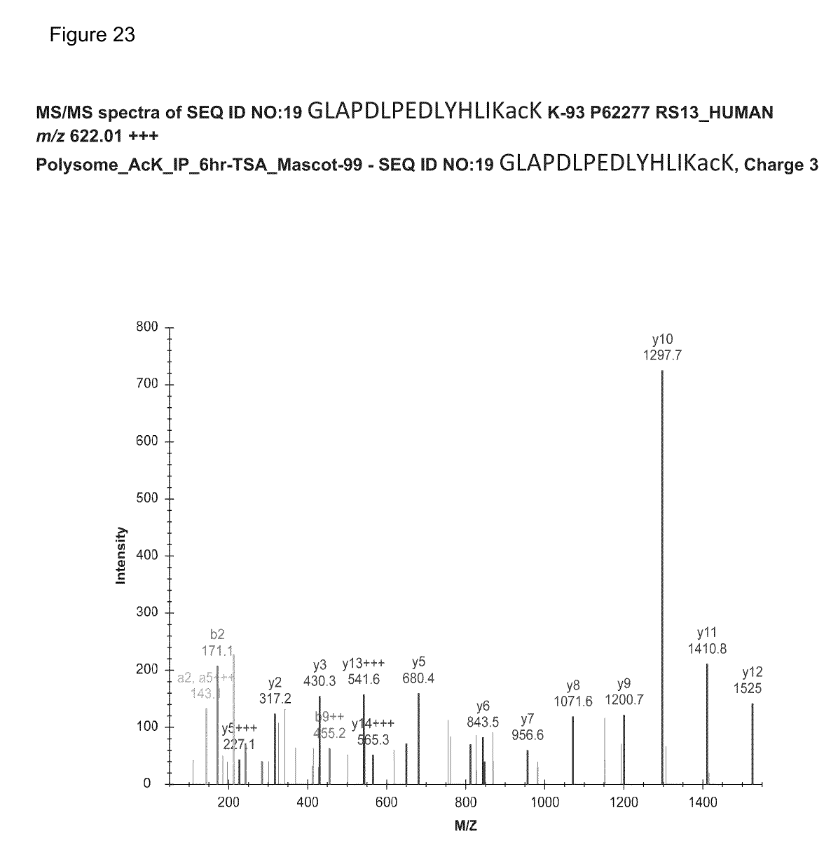

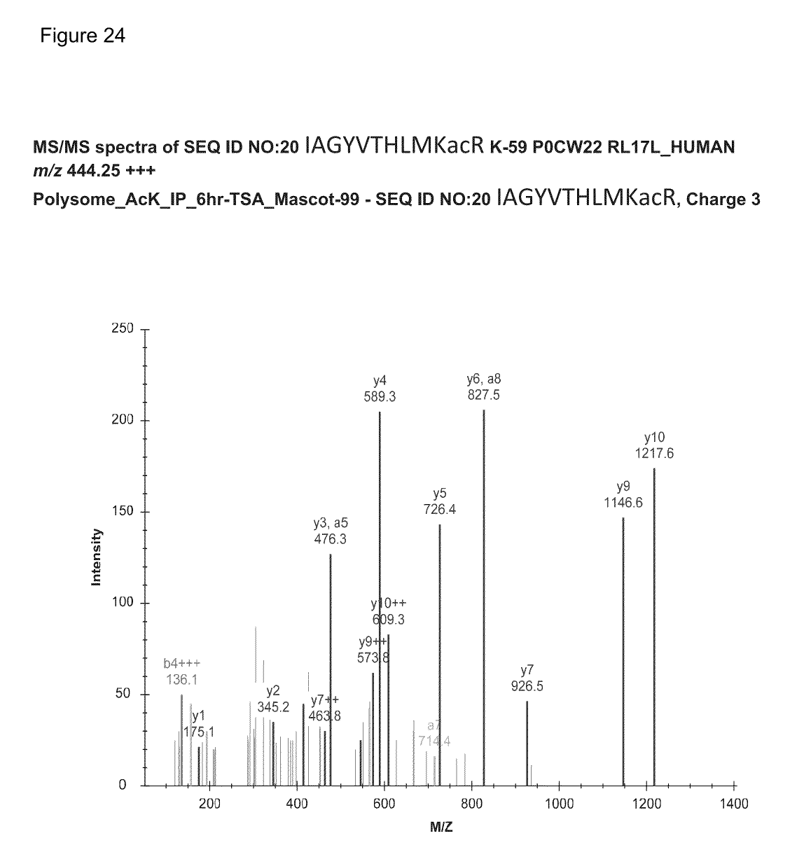

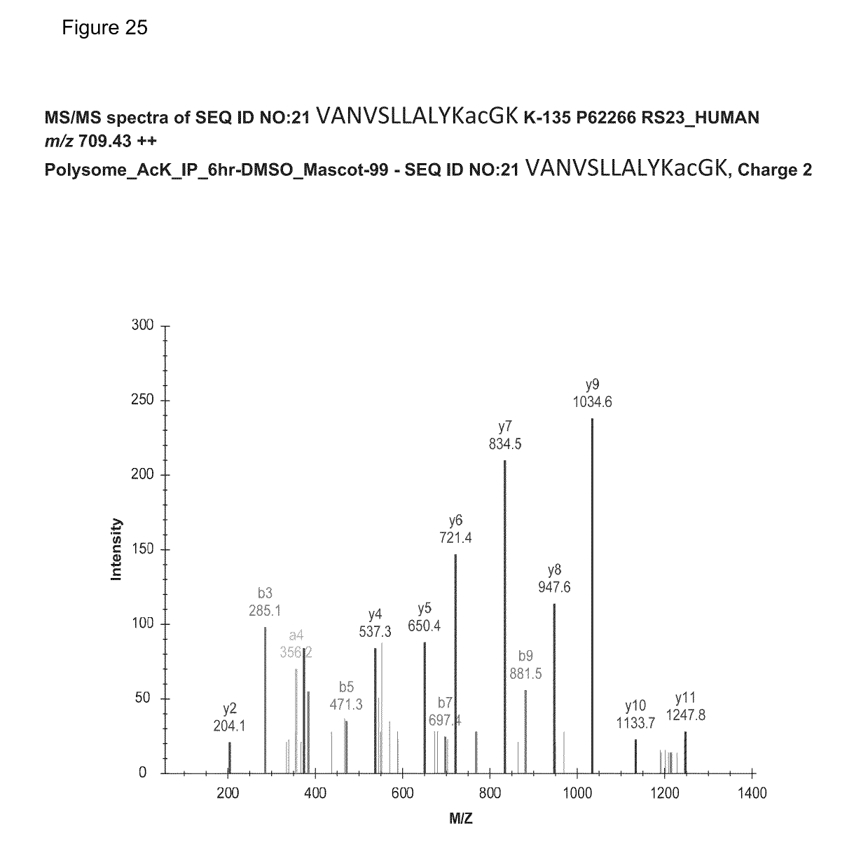

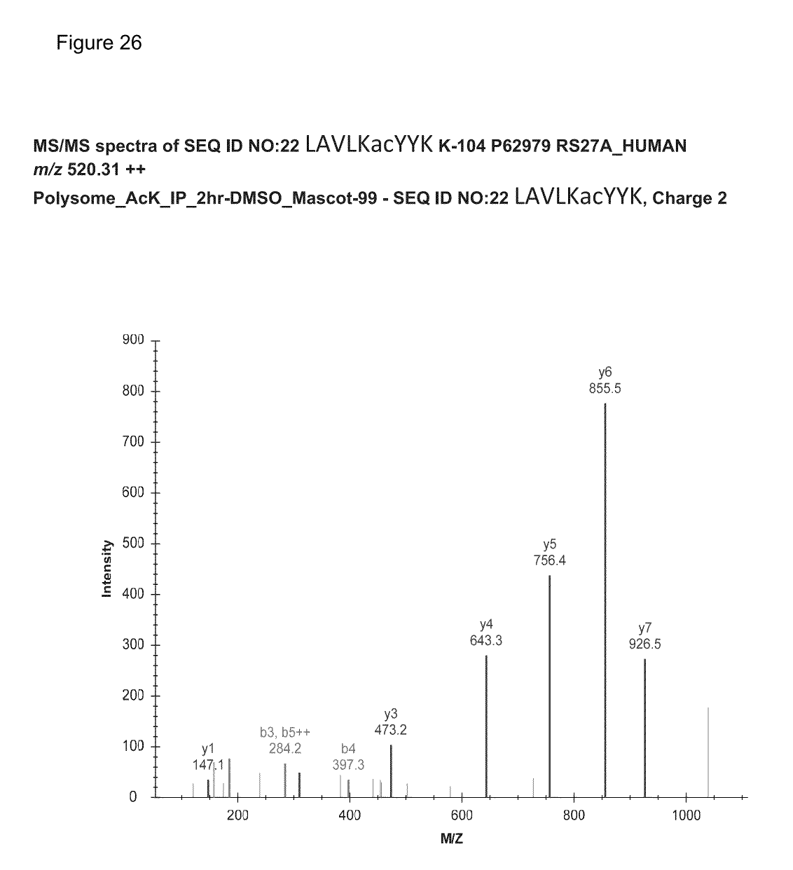

FIGS. 11-26: ESI-MS/MS spectra for lysine acetylated peptides obtained from polysome preparations. For each acetylated peptide the annotated ESI-MS/MS spectrum is displayed, the peptide sequence is indicated including the acetylated lysine residue ‘Kac’ within the sequence, and the lysine acetylation site (K residue number) is provided. In addition, SwissProt accession numbers and the corresponding protein names are listed. The precursor ion m/z value that was selected for MS/MS as well as the charge state are displayed above the spectrum. Fragment ions are annotated as y or b ions within the spectrum above the observed fragment ion m/z values. All spectra were acquired on a quadrupole time-of-flight (QqTOF) TripleTOF 5600 mass spectrometer.

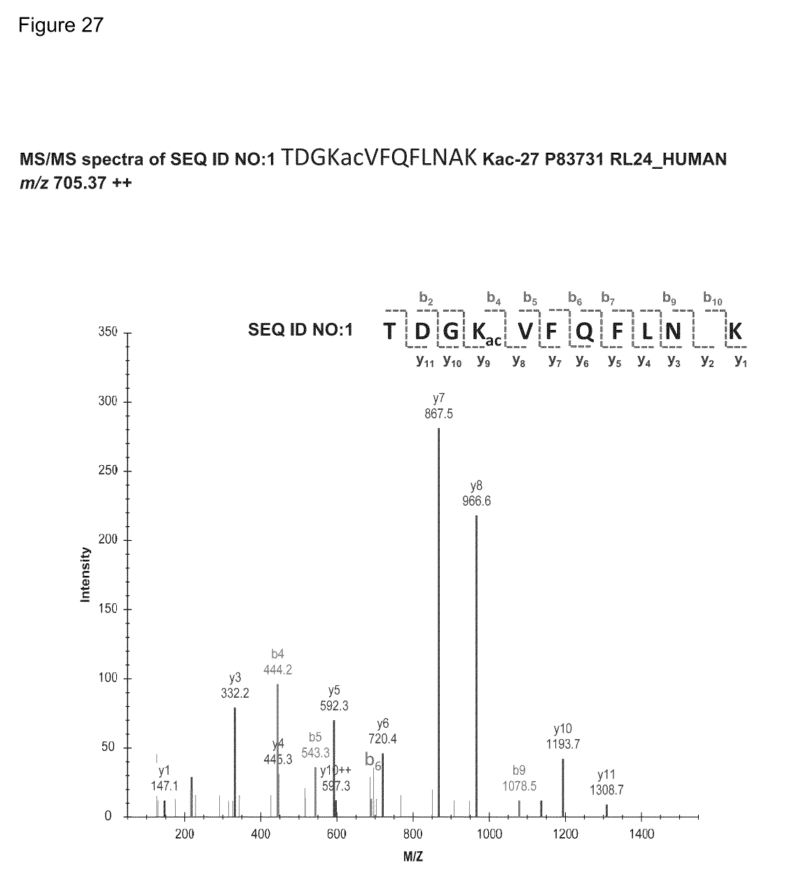

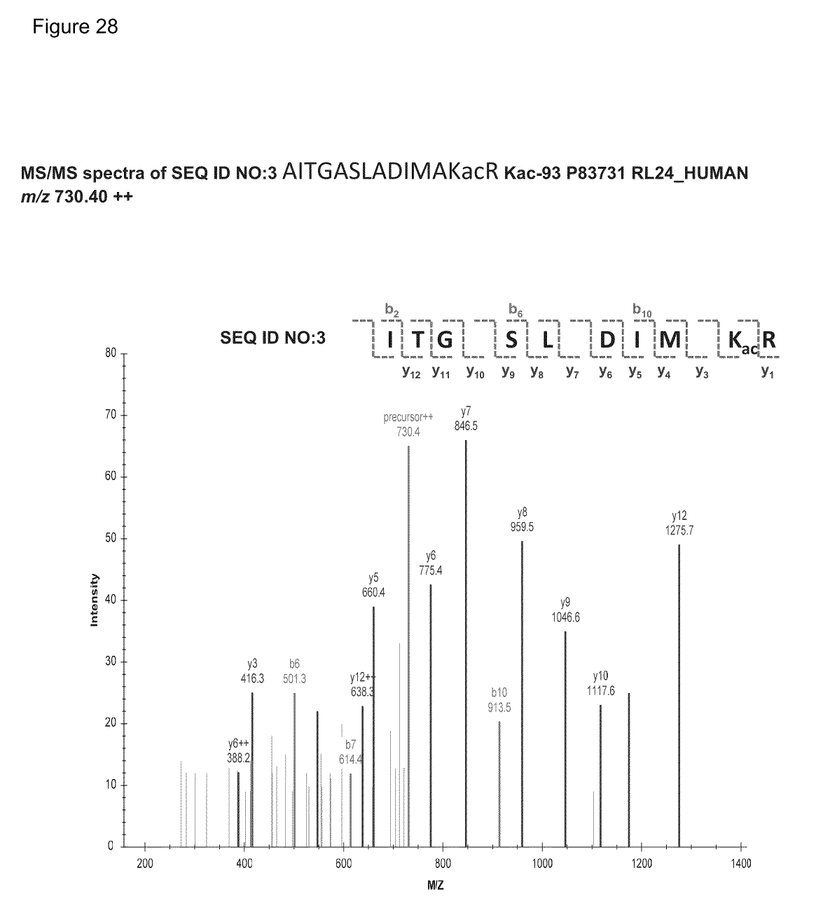

FIGS. 27 and 28: ESI-MS/MS spectra for lysine acetylated peptides obtained from 60S preparations. A representative MS/MS spectra for the acetyl-K27 (FIG. 27) and acetyl-K93 (FIG. 28) sites of 60S-associated RPL24. Peptide [M+2H]²⁺ precursor ions m/z 705.37 and m/z 730.40 were fragmented by collision-induced dissociation (CID). The y-type and b-type ions were used to identify the peptide sequence and locate the acetylation site.

### Synthesis of Compounds

The synthesis of the compounds provided herein can be found below. Compounds provided herein can be conveniently prepared or formed during the processes provided herein, as solvates (e.g., hydrates). Hydrates of compounds provided herein can be conveniently prepared by recrystallization from an aqueous/organic solvent mixture, using organic solvents such as dioxan, tetrahydrofuran or methanol.

In addition, some of the compounds provided herein have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included herein. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds provided herein may also be represented in multiple tautomeric forms, in such instances all tautomeric forms of the compounds described herein are included. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included herein. All crystal forms of the compounds described herein are expressly included herein.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds provided herein. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In embodiments, provided herein are intermediate compounds of the formulae delineated herein and methods of converting such compounds to compounds of the formulae herein (e.g., in schemes herein) comprising reacting a compound herein with one or more reagents in one or more chemical transformations (including those provided herein) to thereby provide the compound of any of the formulae herein or an intermediate compound thereof.

The synthetic methods described herein may also additionally include steps, either before or after any of the steps described in any scheme, to add or remove suitable protecting groups in order to ultimately allow synthesis of the compound of the formulae described herein. The methods delineated herein contemplate converting compounds of one formula to compounds of another formula (e.g., in Scheme A, A1 to A2; A2 to A3; A1 to A3). The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, sublimation, crystallization, trituration, solid phase extraction, and chromatography).

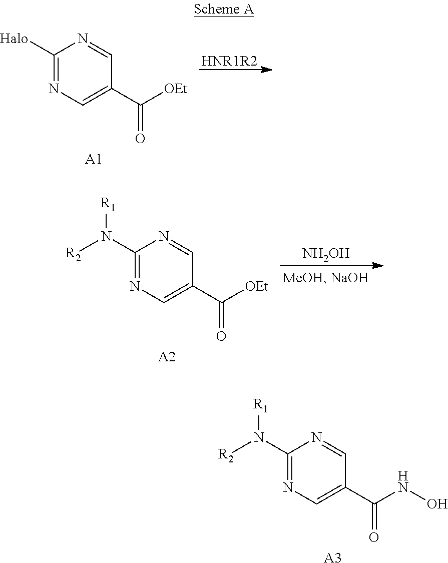

The compounds provided herein may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds provided herein are defined herein by their chemical structures or chemical names, or both. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof. The recitation of an embodiment for a variable herein includes that embodiment as any single embodiment or in combination with any other embodiments or portions thereof.

The syntheses of the compounds of formula (IV) are provided in U.S. patent application Ser. No. 13/296,748 (now U.S. Pat. No. 8,614,223), which is incorporated herein by reference in its entirety.

1. A method for treating a subject diagnosed with an RPL24-overexpressing cancer comprising administering an HDAC inhibitor to the subject in need thereof.

2. The method of claim 1, wherein the HDAC is selected from HDAC1, HDAC2, HDAC3, or HDAC8.

3. The method of claim 1, wherein the HDAC is selected from HDAC4, HDAC5, HDAC6, HDAC7, HDAC9, or HDAC10.

4. The method of claim 1, wherein the HDAC is HDAC11.

5. The method of claim 1, wherein the HDAC is HDAC6.

6. The method of claim 1, wherein the cancer is a lung cancer.

7. The method of claim 1, wherein the cancer is a breast cancer.

8. The method of claim 7, wherein the breast cancer is a basal-like breast cancer.

9. The method of claim 1, wherein the cancer is an Myc-induced cancer.

10. The method of claim 1, wherein the cancer is an Akt-induced cancer.

11-33. (canceled)

34. The method of claim 1, wherein the HDAC inhibitor is a compound of formula IV: 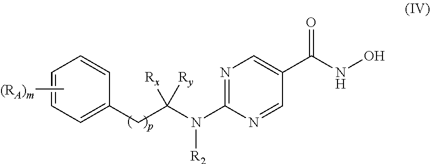
or a pharmaceutically acceptable salt thereof,
wherein,
R₂ is H or alkyl;
Rₓ and R_{y} are independently H, alkyl, or aryl, wherein the alkyl and aryl groups may be substituted with halo; or Rₓ and R_{y} together with the carbon to which each is attached, forms a cycloalkyl or heterocycloalkyl ring;
each R_{A} is independently alkyl, alkoxy, aryl, halo, or haloalkyl; or two R_{A} groups, together with the atoms to which each is attached, can form a heterocycloalkyl ring;
m is 0, 1, or 2; and
p is 0 or 1.

35. The method of claim 34, wherein:
R₂ is H;
Rₓ and R_{y} are independently H, alkyl, aryl, or haloaryl; or Rₓ and R_{y} together with the carbon to which each is attached, forms a cycloalkyl or heterocycloalkyl ring;
each R_{A} is independently alkyl, alkoxy, aryl, halo, or haloalkyl; or two R_{A} groups, together with the atoms to which each is attached, can form a heterocycloalkyl ring;
m is 0, 1, or 2; and
p is 0.

36. The method of claim 34, wherein Rₓ and R_{y}, together with the carbon to which each is attached, forms a cyclopropyl, cyclobutyl, cyclopentyl, cyclohexyl, oxetanyl, or tetrahydropyranyl ring.

37. The method of claim 34, wherein Rₓ and R_{y}, together with the carbon to which each is attached, forms a cyclopropyl, cyclopentyl, cyclohexyl, or tetrahydropyran ring.

38. The method of claim 34, wherein Rₓ and R_{y}, together with the carbon to which each is attached, forms a cyclopropyl or cyclohexyl ring.

39. The method of claim 34, wherein m is 0, 1 or 2, and each R_{A} is independently methyl, phenyl, F, Cl, methoxy, or CF₃; or two R_{A} groups, together with the atoms to which each is attached, form a dioxole ring.

40. The method of claim 34, wherein m is 1, and R_{A} is F, Cl, methoxy, or CF₃.

41. The method of claim 1, wherein the HDAC inhibitor is a compound selected from the following: 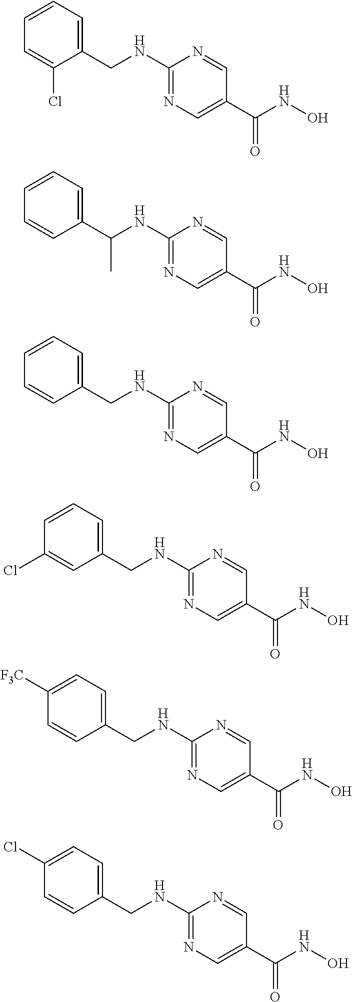 [SMILES: O=C(NO)c1cnc(NCc2ccccc2Cl)nc1 ; CC(Nc1ncc(C(=O)NO)cn1)c1ccccc1 ; O=C(NO)c1cnc(NCc2ccccc2)nc1 ; O=C(NO)c1cnc(NCc2cccc(Cl)c2)nc1 ; O=C(NO)c1cnc(NCc2ccc(C(F)(F)F)cc2)nc1 ; O=C(NO)c1cnc(NCc2ccc(Cl)cc2)nc1] 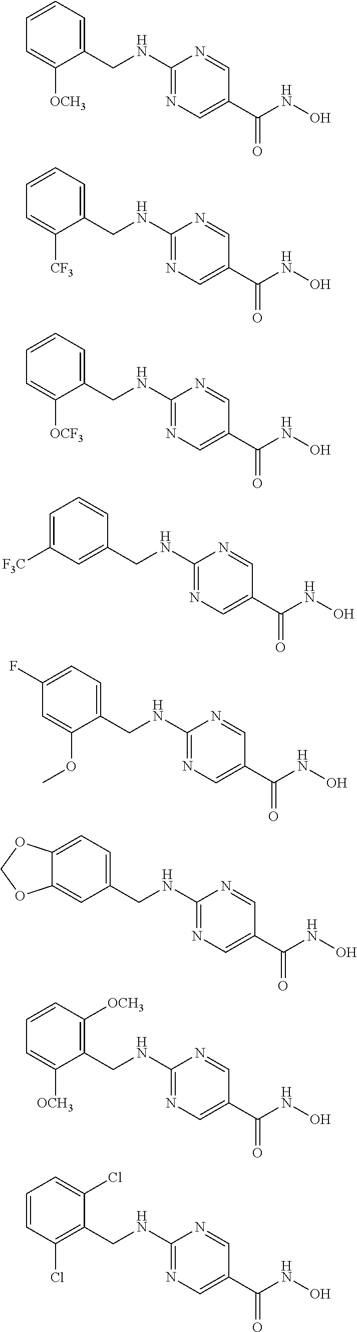 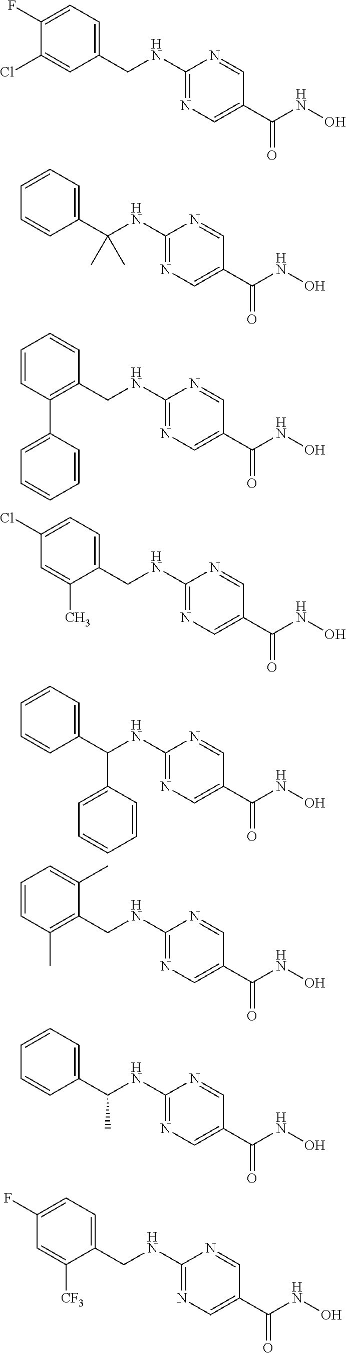 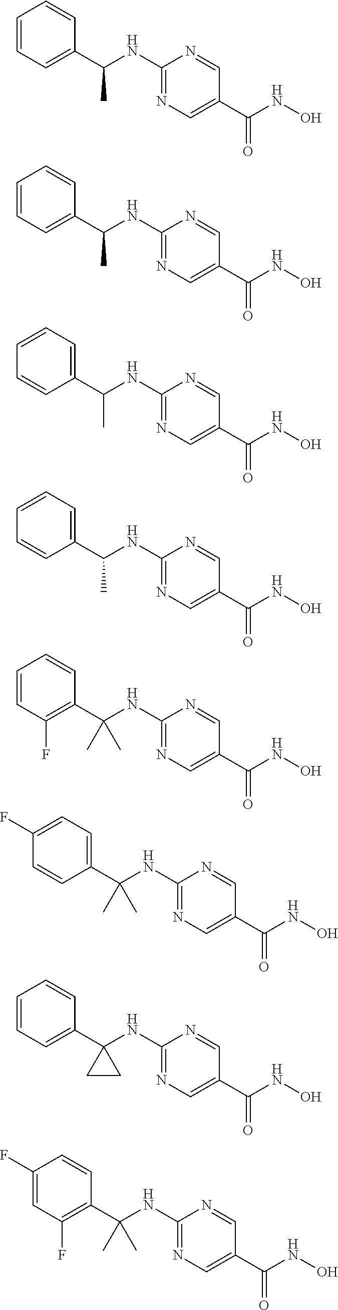 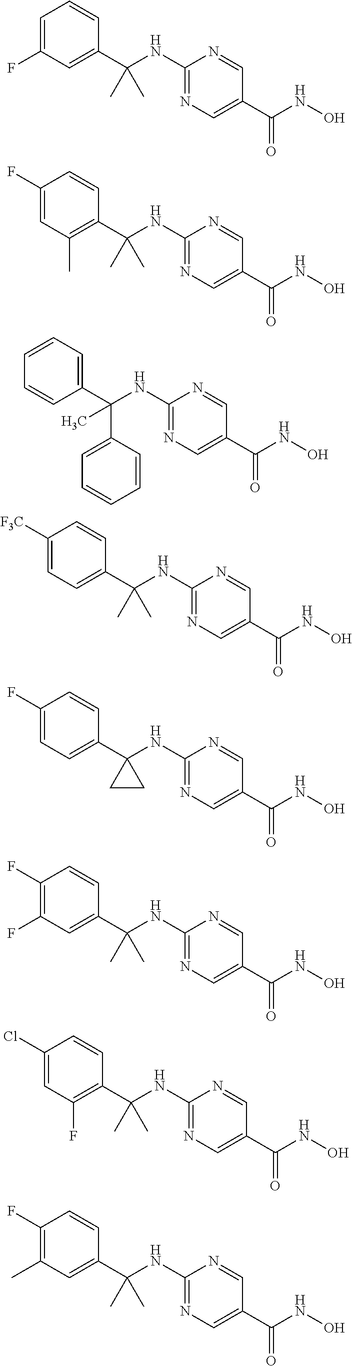 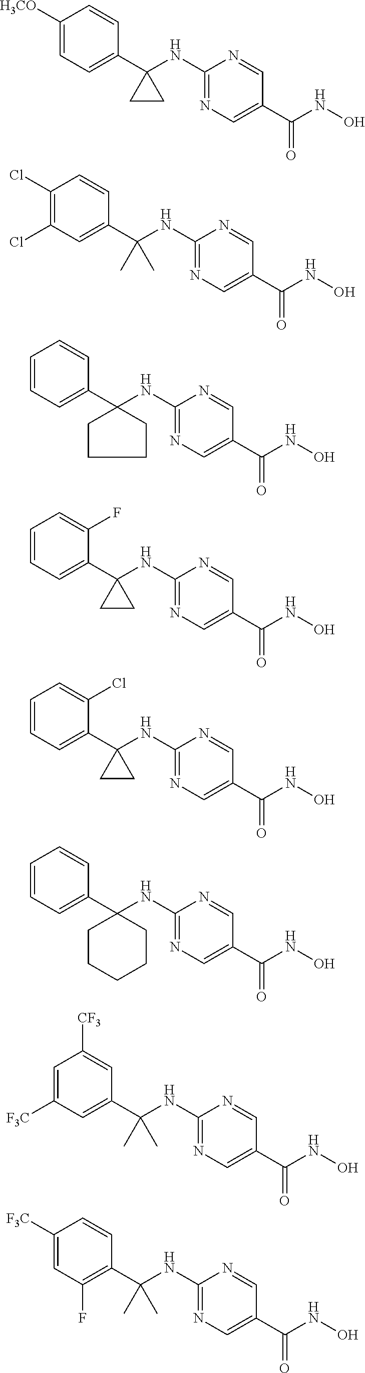 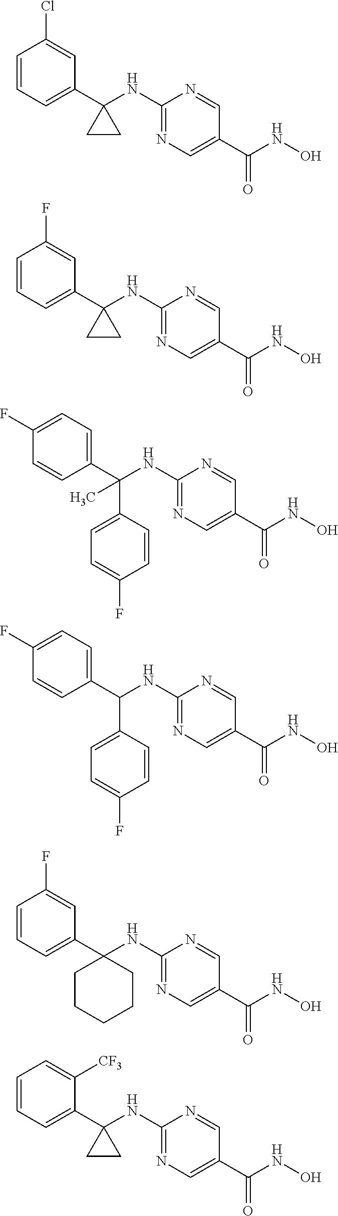 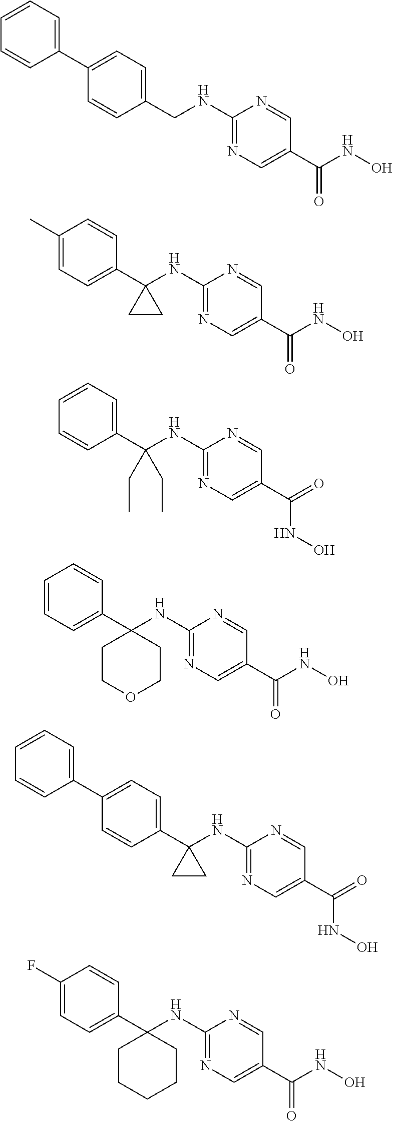 [SMILES: O=C(NO)c1cnc(NCc2ccc(-c3ccccc3)cc2)nc1 ; Cc1ccc(C2(Nc3ncc(C(=O)NO)cn3)CC2)cc1 ; CCC(CC)(Nc1ncc(C(=O)NO)cn1)c1ccccc1 ; O=C(NO)c1cnc(NC2(c3ccccc3)CCOCC2)nc1 ; O=C(NO)c1cnc(NC2(c3ccc(-c4ccccc4)cc3)CC2)nc1 ; O=C(NO)c1cnc(NC2(c3ccc(F)cc3)CCCCC2)nc1] 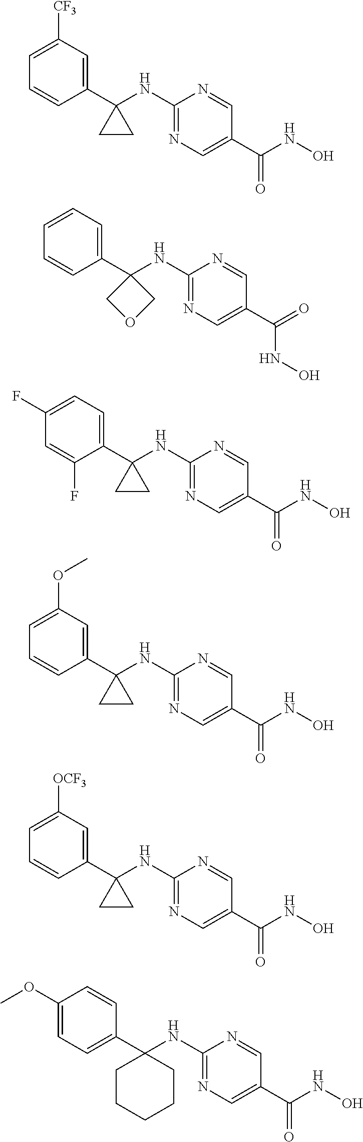 [SMILES: O=C(NO)c1cnc(NC2(c3cccc(C(F)(F)F)c3)CC2)nc1 ; O=C(NO)c1cnc(NC2(c3ccccc3)COC2)nc1 ; O=C(NO)c1cnc(NC2(c3ccc(F)cc3F)CC2)nc1 ; COc1cccc(C2(Nc3ncc(C(=O)NO)cn3)CC2)c1 ; O=C(NO)c1cnc(NC2(c3cccc(OC(F)(F)F)c3)CC2)nc1 ; COc1ccc(C2(Nc3ncc(C(=O)NO)cn3)CCCCC2)cc1] 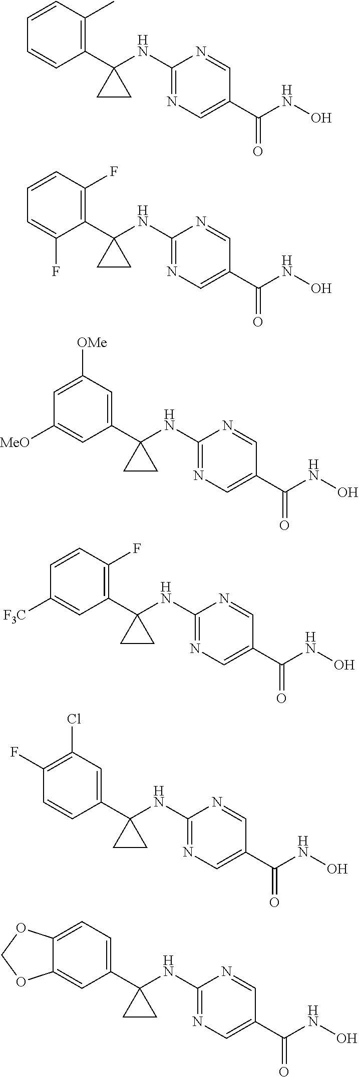 [SMILES: Cc1ccccc1C1(Nc2ncc(C(=O)NO)cn2)CC1 ; O=C(NO)c1cnc(NC2(c3c(F)cccc3F)CC2)nc1 ; COc1cc(OC)cc(C2(Nc3ncc(C(=O)NO)cn3)CC2)c1 ; O=C(NO)c1cnc(NC2(c3cc(C(F)(F)F)ccc3F)CC2)nc1 ; O=C(NO)c1cnc(NC2(c3ccc(F)c(Cl)c3)CC2)nc1 ; O=C(NO)c1cnc(NC2(c3ccc4c(c3)OCO4)CC2)nc1] 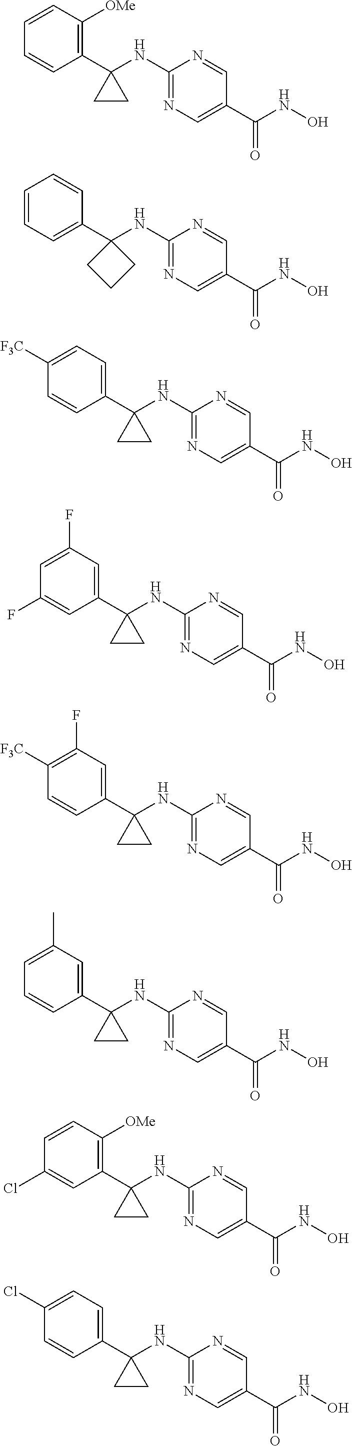 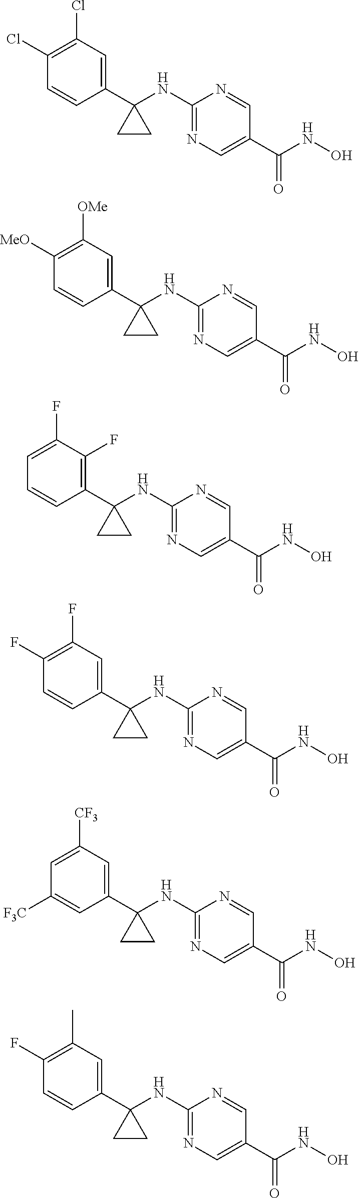 [SMILES: O=C(NO)c1cnc(NC2(c3ccc(Cl)c(Cl)c3)CC2)nc1 ; COc1ccc(C2(Nc3ncc(C(=O)NO)cn3)CC2)cc1OC ; O=C(NO)c1cnc(NC2(c3cccc(F)c3F)CC2)nc1 ; O=C(NO)c1cnc(NC2(c3ccc(F)c(F)c3)CC2)nc1 ; O=C(NO)c1cnc(NC2(c3cc(C(F)(F)F)cc(C(F)(F)F)c3)CC2)nc1 ; Cc1cc(C2(Nc3ncc(C(=O)NO)cn3)CC2)ccc1F] 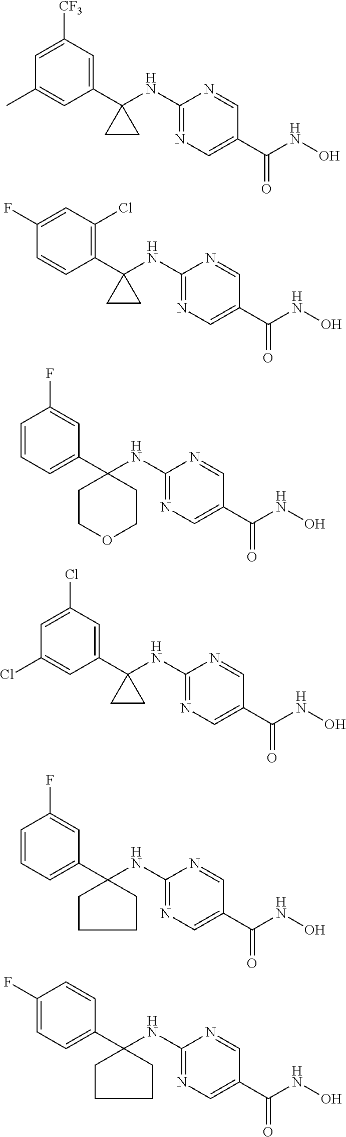 [SMILES: Cc1cc(C(F)(F)F)cc(C2(Nc3ncc(C(=O)NO)cn3)CC2)c1 ; O=C(NO)c1cnc(NC2(c3ccc(F)cc3Cl)CC2)nc1 ; O=C(NO)c1cnc(NC2(c3cccc(F)c3)CCOCC2)nc1 ; O=C(NO)c1cnc(NC2(c3cc(Cl)cc(Cl)c3)CC2)nc1 ; O=C(NO)c1cnc(NC2(c3cccc(F)c3)CCCC2)nc1 ; O=C(NO)c1cnc(NC2(c3ccc(F)cc3)CCCC2)nc1] 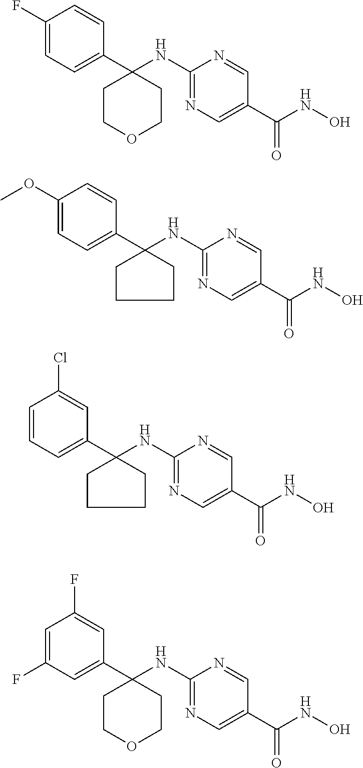 [SMILES: O=C(NO)c1cnc(NC2(c3ccc(F)cc3)CCOCC2)nc1 ; COc1ccc(C2(Nc3ncc(C(=O)NO)cn3)CCCC2)cc1 ; O=C(NO)c1cnc(NC2(c3cccc(Cl)c3)CCCC2)nc1 ; O=C(NO)c1cnc(NC2(c3cc(F)cc(F)c3)CCOCC2)nc1]
or a pharmaceutically acceptable salt thereof.


SEQUENCE LISTING (22 sequences)

SEQ ID NO: 1 - protein, 12 aa, Homo sapiens
  MOD_RES (4)..(4): ACETYLATION
TDGKVFQFLNAK

SEQ ID NO: 2 - protein, 14 aa, Homo sapiens
  MOD_RES (11)..(11): OXIDIZED
AITGASLADIMAKR

SEQ ID NO: 3 - protein, 14 aa, Homo sapiens
  MOD_RES (13)..(13): ACETYLATION
AITGASLADIMAKR

SEQ ID NO: 4 - DNA, 21 bp, Artificial Sequence
  TARGET SEQUENCE
cctgaagttagaaaggctcaa

SEQ ID NO: 5 - DNA, 21 bp, Artificial Sequence
  TARGET SEQUENCE
gtgcatctcttgctgatataa

SEQ ID NO: 6 - DNA, 21 bp, Artificial Sequence
  TARGET SEQUENCE
tacaacagccacaacgtctat

SEQ ID NO: 7 - protein, 11 aa, Homo sapiens
  MOD_RES (9)..(9): ACETYLATION
HMYHSLYLKVK

SEQ ID NO: 8 - protein, 10 aa, Homo sapiens
  MOD_RES (8)..(8): ACETYLATION
ILMEHIHKLK

SEQ ID NO: 9 - protein, 12 aa, Homo sapiens
  MOD_RES (11)..(11): ACETYLATION
ITVTSEVPFSKR

SEQ ID NO: 10 - protein, 10 aa, Homo sapiens
  MOD_RES (9)..(9): ACETYLATION
DVFRDPALKR

SEQ ID NO: 11 - protein, 8 aa, Homo sapiens
  MOD_RES (7)..(7): ACETYLATION
GVVVVIKR

SEQ ID NO: 12 - protein, 13 aa, Homo sapiens
  MOD_RES (12)..(12): ACETYLATION
TVFAEHISDECKR

SEQ ID NO: 13 - protein, 10 aa, Homo sapiens
  MOD_RES (9)..(9): ACETYLATION
DETEFYLGKR

SEQ ID NO: 14 - protein, 10 aa, Homo sapiens
  MOD_RES (6)..(6): ACETYLATION
VGIVGKYGTR

SEQ ID NO: 15 - protein, 12 aa, Homo sapiens
  MOD_RES (7)..(7): ACETYLATION
FIDTTSKFGHGR

SEQ ID NO: 16 - protein, 12 aa, Homo sapiens
  MOD_RES (4)..(4): ACETYLATION
THQKFVIATSTK

SEQ ID NO: 17 - protein, 12 aa, Homo sapiens
  MOD_RES (11)..(11): ACETYLATION
NFGIGQDIQPKR

SEQ ID NO: 18 - protein, 14 aa, Homo sapiens
  MOD_RES (13)..(13): ACETYLATION
AGVNTVTTLVENKK

SEQ ID NO: 19 - protein, 16 aa, Homo sapiens
  MOD_RES (15)..(15): ACETYLATION
GLAPDLPEDLYHLIKK

SEQ ID NO: 20 - protein, 11 aa, Homo sapiens
  MOD_RES (10)..(10): ACETYLATION
IAGYVTHLMKR

SEQ ID NO: 21 - protein, 13 aa, Homo sapiens
VANVSLLALYKGK

SEQ ID NO: 22 - protein, 8 aa, Homo sapiens
  MOD_RES (5)..(5): ACETYLATION
LAVLKYYK